(19) United States
(12) Patent Application Publication
(10) Pub. No.: US 2020/0172864 A1
(43) Pub. Date: Jun. 4, 2020

(54) T Cell Expansion Method

(71) Applicants: TESSA THERAPEUTICS PTE. LTD., Singapore (SG); SINGAPORE HEALTH SERVICES PTE. LTD., Singapore (SG)

(72) Inventors: Cheryl Lai-Lai CHIANG, Singapore (SG); Who-Whong WANG, Singapore (SG); Han Chong TOH, Singapore (SG)

(73) Assignees: Tessa Therapeutics Ltd., Singapore (SG); Singapore Health Services Pte. Ltd., Singapore (SG)

(21) Appl. No.: 16/336,058

(22) PCT Filed: Sep. 26, 2017
(86) PCT No.: PCT/EP2017/074331

§ 371 Date: Mar. 22, 2019

Related U.S. Application Data
(60) Provisional application No. 62/399,604, filed on Sep. 26, 2016.

(51) Int. Cl. C12N 5/0783; A61K 35/17

(52) U.S. Cl. CPC C12N 5/0636; A61K 35/17; C12N 2506/11; C12N 2501/2307; C12N 2501/2308; C12N 2501/2315; C12N 2501/2321; C12N 2501/2302

(57) ABSTRACT

The invention relates to the expansion of T cells and particularly, although not exclusively, to the expansion of gamma delta T cells, and the optimization of medium, serum and cytokine combinations for large-scale ex vivo expansion of gamma delta T cells for clinical use.

### FIELD OF THE INVENTION

The present invention relates to the expansion of T cells and particularly, although not exclusively, to the expansion of gamma delta T cells.

### BACKGROUND TO THE INVENTION

Human gamma delta (γδ) T cells are a heterogeneous population of immune cells that consists of three major subtypes—i.e. Vδ1, Vδ2 and Vδ3, residing in specific anatomical sites. The Vδ1 subtype is present in the epithelial, dermis, liver and spleen, Vδ2 is found in the peripheral blood while Vδ3 resides in the liver and gut epithelium (reviewed in 1). The Vδ2 subtype makes up 1-5% of the peripheral blood lymphocyte (PBL) population and >90% of the Vδ2 subtype preferentially expresses the Vγ9Vδ2 T cell receptor (TCR). The Vγ9Vδ2 T cells are the most widely used population of γδ T cells for tumor immunotherapy as they are easily obtained from the peripheral blood for large-scale expansion.

Unlike the αβ CD4⁺ and CD8⁺ T cells that are well-studied, γδ T cells are discovered nearly 20 years ago and their specific functions in the immune system remain elusive. However, studies have shown that Vγ9Vδ2 T cells played an important protective role against environmental stress and pathogen infections. During infancy, the Vγ9Vδ2 T cells are present in low numbers but are preferentially expanded in response to environmental stimuli (2). In adulthood, Vγ9Vδ2 T cells are rapidly expanded in microbial infections (3). Vγ9Vδ2 T cells also play a potent role in tumor surveillance. They recognize isopentenyl pyrophosphate (IPP) [4,5] which is an intermediate metabolite in the mevalonate pathway that is increased in some malignant cells and in almost all cells upon pharmacological treatment with bisphosphonates (6,7) or alkyl amines (3). In addition, Vγ9Vδ2 T cells directly respond to a variety of stress-induced self-antigens (e.g. MICA, MICB, ULBP and heat-shock proteins) expressed by malignant cells (8). Upon activation, Vγ9Vδ2 T cells effectively lyse a broad range of tumor cells, including leukemia cells, nasopharyngeal carcinoma (8), breast carcinoma (9), hepatocellular carcinoma (10), lung carcinoma (11), renal cell carcinoma (12, 13), pancreatic adenocarcinoma (14), prostate carcinoma (15), and neuroblastoma (16). Moreover, ex vivo generated Vγ9Vδ2 T cells that are adoptively transferred into various tumor xenograft mouse models showed anti-tumor activities in vivo (8, 14, 17). Hence, these findings strongly supported the rationale of using Vγ9Vδ2 T cells to target cancers.

Indeed, clinical trials have been performed to harness the anti-tumor properties of Vγ9Vδ2 T cells. One study administered pamidronate, which is a bisphosphate drug, and interleukin (IL)-2 to patients with refractory or relapsing B cell malignancies to stimulate the proliferation of Vγ9Vδ2 in vivo [18]. This resulted in the partial remission and stable diseases in some patients. In another study, patients with metastatic prostate carcinoma were given bisphosphonate, zoledronic acid (another drug to accumulate IPP, isopentenyl pyrophosphate) and IL-2. Some patients underwent partial remission or stable disease following treatment. Notably, in those patients with the highest numbers of circulating Vγ2Vδ2 T cells they also showed the lowest levels of prostate-specific antigen tumor cell marker (19). Other clinical trials have been performed to treat prostate or renal carcinoma patients with ex vivo expanded Vγ9Vδ2 T cells (reviewed in 20). Encouraging results were observed and some patients achieved partial remission or stable disease. This further demonstrated the promising potential of Vγ9Vδ2 T cell-based immunotherapy against cancers.

Ex vivo expansion and administration of Vγ9Vδ2 T cells to cancer patients is a more straightforward approach compared to in vivo Vγ9Vδ2 T cell activation with bisphosphate drug and cytokine administration. The main advantage of ex vivo expansion method is that Vγ9Vδ2 T cells could be propagated to a large number before infusing them into the patients. Moreover, the cells could be manipulated ex vivo to maximize their anti-tumor properties, and the quality of the generated cells could be controlled before administration.

### SUMMARY OF THE INVENTION

The present invention relates to gamma delta T cells, and methods for generating and expanding gamma delta T cells. Gamma delta T cells may be generated from PBMCs in T cell media comprising one or more cytokines and optionally serum. Preferably, the one or more cytokines are interleukins. Thus, one gamma delta T cell culture may comprise one, two, three or more interleukins. The culture may additionally comprise one or more cytokines that are not interleukins.

Gamma delta T cells generated/expanded in accordance with the methods described herein are provided with particularly advantageous properties and are useful in methods to treatment, and also in methods for expanding antigen-specific T cells.

Described herein is a method for generating or expanding gamma delta T cells, the method comprising culturing peripheral blood mononuclear cells (PBMCs) in the presence of IL2 and IL21, or IL2 and IL18.

Also described is a method for generating or expanding gamma delta T cells, the method comprising culturing PBMCs in the presence of IL15. Optionally, the method comprises culturing the PBMCs in the presence of IL15 and IL21. Optionally, the PBMCs are cultured in the presence of IL15, IL 21 and IL18.

Also described is a method for generating or expanding gamma delta T cells, the method comprising culturing PBMCs in the presence of IL21. The method may comprise culturing the PBMCs in the presence of IL21 and IL2 and/or IL15.

In some methods described herein, the PBMCs have been obtained from a sample of human peripheral blood.

The gamma delta T cells may be Vδ2 T cells. They may be Vγ9Vδ2 T cells.

In some methods disclosed herein, the PBMCs are cultured in culture medium supplemented with serum. The serum may be human serum. The culture medium may be supplemented with 10% serum. The medium may be OpTimizer T cell media. The serum may be human AB serum, such as pooled human AB serum. The serum may be defined FBS.

Methods disclosed herein may generate a population of cells which comprises at least 60% gamma delta T cells, preferably at least 70% gamma delta T cells. Also disclosed herein is an isolated population of cells that comprises at least 60% gamma delta T cells, preferably at least 70% gamma delta T cells.

Also disclosed herein are gamma delta T cells generated, expanded and obtained from, or obtainable from methods disclosed herein. Gamma delta T cells disclosed herein may exhibit antigen presentation and/or effector phenotypes.

Gamma delta T cells disclosed herein may express a higher level of at least one marker selected from HLA-ABC, HLA-DR, CD80, CD83, CD86, CD40 and ICAM-1 than has been generated in the presence of IL2 alone.

Gamma delta T cells disclosed herein may express a higher level of at least one marker selected from CCR5, CCR6, CCR7, CD27 and NKG2D than a gamma delta T cell that has been generated in the presence of IL2 alone.

Gamma delta T cells disclosed herein may be used in medicine. The cells may be useful in methods of adoptive T cell therapy, such as autologous T cell therapy.

The present disclosure also provides a cell culture comprising gamma delta T cells, media, and cytokines, wherein the cytokines are selected from:
IL2 and IL21;
IL15;
IL15 and IL21;
IL12 and IL18;
IL15, IL18 and IL21.
IL2 and IL7;
IL2 and IL15;
IL2, IL18 and 1121;
IL15 and IL7; or
IL15 and IL18.

The cell culture may also comprise serum, preferably 10% serum.

Also disclosed herein is a method for generating or expanding a population of antigen-specific T cells, comprising stimulating T cells by culture in the presence of gamma delta T cells generated/expanded according to the method of the present invention presenting a peptide of the antigen, and antigen-specific T cells generated according to such methods. Also disclosed is the use of antigen-specific T cells generated according to such methods in medicine, and adoptive T cell therapy.

Also disclosed herein is a method of treating or preventing a disease or disorder in a subject, comprising:
(a) isolating PBMCs from a subject;
(b) generating or expanding a population of gamma delta T cells according to the method of the present invention, and;
(c) administering the gamma delta T cells to a subject.

Also disclosed herein a method of treating or preventing a disease or disorder in a subject, comprising:
(a) isolating PBMCs from a subject;
(b) generating or expanding a population of gamma delta T cells according to the method of the present invention;
(c) generating or expanding a population of antigen-specific T cells by a method comprising stimulating T cells by culture in the presence of gamma delta T cells generated/expanded according to (b) presenting a peptide of the antigen; and
(d) administering the antigen-specific T cells to a subject.

### DESCRIPTION

There is no standard procedure for large-scale ex vivo expansion of Vγ9Vδ2 T cells for clinical use. All the Vγ9Vδ2 T cell clinical trials published used different concentrations of IL-2 and zoledronic acid for expansion for 10 to 30 days before infusion (reviewed in 20). Also, there was no established parameters for evaluating the anti-tumor properties of the generated Vγ9Vδ2 T cells. Thus, we sought to optimize the medium, serum and cytokine combinations for large-scale ex vivo expansion of Vγ9Vδ2 T cells for clinical use. We also examined and defined parameters for optimal Vγ9Vδ2 T cell generation and potency assessment by assessing their phenotype, cytokine profile, direct tumor cytolysis and antigen presentation for activating anti-tumor CD4⁺ and CD8⁺ T cells.

The invention therefore relates to the inventors investigation of the ability of cytokines, and particularly interleukins, in supporting or enhancing the proliferation of gamma delta T cells, thereby producing a population of cells that is enriched for gamma delta T cells.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Methods for Generating or Expanding Gamma Delta T Cells

Methods disclosed herein are useful for generating or expanding gamma delta T cells. Methods disclosed herein are performed in vitro.

The present invention provides a method for generating or expanding gamma delta T cells comprising culturing a population of immune cells comprising at least one gamma delta T cell in the presence of specific cytokines or combinations thereof. Aspects of the present invention provide methods for generating or expanding gamma delta T cells comprising culturing a population of immune cells comprising at least one gamma delta T cell in the presence of: (i) IL2 and IL21, (ii) IL2 and IL18 (iii) IL15, or (iv) IL21.

Culture of cells in accordance with the methods of the invention is performed using suitable medium and under suitable environmental conditions (e.g. temperature, pH, humidity, atmospheric conditions, agitation etc.) for the in vitro culture of immune cells, which are well known to the person skilled in the art of cell culture.

Conveniently, cultures of cells may be maintained at 37° C. in a humidified atmosphere containing 5% CO₂. Cultures can be performed in any vessel suitable for the volume of the culture, e.g. in wells of a cell culture plate, cell culture flasks, a bioreactor, etc. The cell cultures can be established and/or maintained at any suitable density, as can readily be determined by the skilled person. For example, cultures may be established at an initial density of ˜0.5×10⁶ to ˜5×10⁶ cells/ml of the culture (e.g. ˜1×10⁶ cells/ml). Cells may be cultured in any suitable cell culture vessel. In some embodiments of the methods according to the various aspects of the present invention, cells are cultured in a bioreactor. In some embodiments, cells are cultured in a bioreactor described in Somerville and Dudley, Oncoimmunology (2012) 1(8):1435-1437, which is hereby incorporated by reference in its entirety. In some embodiments cells are cultured in a GRex cell culture vessel, e.g. a GRex flask or a GRex 100 bioreactor.

Methods disclosed herein may be used to generate gamma delta T cells. In some embodiments, Gamma delta T cells may be generated or expanded from a population of immune cells. It will be appreciated that the population of immune cells comprises gamma delta T cells, e.g. at low frequency. The population of immune cells from which gamma delta T cells are generated/expanded according to the methods of the present invention comprise at least one gamma delta T cell.

Gamma delta T cells may be generated from PBMCs. The methods may involve expansion of gamma delta T cells (e.g. a population of gamma delta T cells) from within a population of immune cells (e.g. PBMCs, PBLs). For example, a population of gamma delta T cells may be generated/expanded from within a population of immune cells (e.g. PBMCs, PBLs), by culture of the immune cells under conditions causing the activation and/or proliferation of gamma delta T cells within the population. The immune cells (e.g. PBMCs, PBLs) used in the methods of the invention may be freshly obtained, or may be thawed from a sample of immune cells which has previously been obtained and frozen.

In embodiments of the methods disclosed herein, generation or expansion of gamma delta T cells may involve culture of a population of PBMCs. In some embodiments, a population of gamma delta T cells may be generated/expanded from within a population of T cells (e.g. a population of T cells of heterogeneous type and/or specificity), which may have been obtained from a blood sample or a population of PBMCs. Culture of the population of immune cells from which the gamma delta T cells are generated/expanded may result in an increase the number of gamma delta T cells, and/or result in an increased proportion of such cells in the cell population at the end of the culture.

Conditions causing the activation and/or proliferation of gamma delta T cells may cause the preferential activation/proliferation of gamma delta T cells, e.g. over other cells of the population of immune cells from within which the population of gamma delta T cells is generated/expanded. In some embodiments, culture under conditions causing the activation and/or proliferation of gamma delta T cells comprises culture in the presence of an agent capable of stimulating the proliferation of gamma delta T cells.

Agents capable of stimulating the proliferation of gamma delta T cells include zoledronic acid and pamidronate (see e.g. Kobayashi and Tanaka Pharmaceuticals (Basel). 2015 March; 8(1): 40-61, which is hereby incorporated by reference in its entirety). Gamma delta T cells can be activated by phospho antigens and aminobisphosphonates. They may be generated by exposing PBMCs to phospho antigens and aminobisphosphonates. Zoledronic acid is a bisphosphonate drug that may be used to activate gamma delta T cells from PBMCs. Pamidronate is another bisphosphonate drug which may be used to activate gamma delta T cells. Some methods disclosed herein additionally involve culturing PBMCs in the presence of a phosphoantigen or aminobisphosphonate, such as zoledronic acid or pamidronate.

In the methods of the present disclosure, agents capable of stimulating the proliferation of gamma delta T cells may be provided to the cell culture in an amount (i.e. at a concentration) sufficient to stimulate the proliferation of gamma delta T cells present in the culture. Suitable amounts/concentrations and timings for adding such agents to the culture can be readily determined by the skilled person, according to the particular agent. By way of example, in the experimental examples of the present application, 5 μM zoledronic acid is added to cultures of PBMCs on days 1 and 3 of the culture.

In some embodiments, the method comprises culture in the presence of an agent capable of stimulating the proliferation of gamma delta T cells. In some embodiments the agent is an agent capable of preferentially stimulating the proliferation of gamma delta T cells, e.g. over proliferation of other immune cells (e.g. ap T cells). In some embodiments the method comprises culture in the presence of a phosphoantigen and/or aminobisphosphonate. In some embodiments the method comprises culture in the presence of zoledronic acid and/or pamidronate. In some embodiments the method comprises culture in the presence of zoledronic acid. In some embodiments zoledronic acid is added to the culture at a final concentration (i.e. a concentration in the culture) of one of 0.5-20 μM, 1-15 μM, 2-10 μM, or 3-8 μM. In some embodiments zoledronic acid is added to the culture at a final concentration of 1 μM, 2 μM, 3 μM, 4 μM, 5 μM, 6 μM, 7 μM, 8 μM, 9 μM or 10 μM.

In some embodiments zoledronic acid is added to the culture on one or more of days 1, 2, 3, 4, 5, 6, 7, 8, 9 and 10. In some embodiments zoledronic acid is added to the culture on day 1 of the culture. In some embodiments zoledronic acid is added to the culture on day 3 of the culture. In some embodiments zoledronic acid is added to the culture on days 1 and 3 of the culture. In some embodiments zoledronic acid is added to the culture: daily, every 2 days, every 3 days, every 4 days or every 5 days.

In some embodiments the agent capable of stimulating the proliferation of gamma delta T cells is added prior to, or at the same time as, adding one or more interleukins to the culture.

In some methods disclosed herein PBMCs are obtained from a sample of peripheral blood and cultured in the presence of one or more cytokines for sufficient time to allow the expansion of gamma delta T cells. Methods may involve the culture of PBMCs for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days or more. Some methods involve the culture of PBMCs for at least 10 days. Methods may involve the culture of PBMCs for 1 to 30 days, 1 to 25 days, 1 to 20 days, 1 to 15 days, 1 to 10 days, 2 to 30 days, 2 to 25 days, 2 to 20 days, 2 to 15 days, 2 to 10 days, 3 to 30 days, 3 to 25 days, 3 to 20 days, 3 to 15 days, 3 to 10 days, 4 to 30 days, 4 to 25 days, 4 to 20 days, 4 to 15 days, 4 to 10 days, 5 to 30 days, 5 to 25 days, 5 to 20 days, 5 to 15 days, or 5 to 10 days.

Certain methods disclosed herein may be used to generate a population of cells that comprises at least 25%, at least 30%, at least 35%, at least 40%, at least 45%, at least 50%, at least 55%, at least 60%, at least 65%, at least 70%, at least 75%, at least 80%, at least 85%, at least 90% or at least 95% gamma delta T cells. Preferably, a population of cells is generated that comprises at least 50%, at least 55% %, at least 60%, at least 65%, at least 70%, at least 75%, at least 80%, at least 85%, at least 90% or at least 95% gamma delta T cells. Also disclosed herein is an isolated population of T cells generated from PBMCs that comprises at least 70%, at least 75% or at least 80% gamma delta T cells. In some cases, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% gamma delta T cells. Preferably, a population of cells is generated that comprises at least 50%, at least 55% %, at least 60%, at least 65%, at least 70%, at least 75%, at least 80%, at least 85%, at least 90% or at least 95% of the T cells in the population are gamma delta T cells.

Some methods disclosed herein may be used to expand a population of gamma delta T cells. Certain methods may result in at least 4×10⁶, at least 5×10⁶, at least 6×10⁶, at least 7×10⁶, at least 8×10⁶, at least 9×10⁶ or at least 10×10⁶ gamma delta T cells being generated from a population of 10 million PBMCs after 10 days in culture. In preferred methods, a population of 10 million PBMCs after 10 days in culture produces a population of at least 10×10⁶, at least 11×10⁶, at least 12×10⁶, at least 13×10⁶, at least 14×10⁶, at least 15×10⁶, at least 16×10⁸, at least 17×10⁶, at least 18×10⁸, at least 19×10⁸ or at least 20×10⁸ gamma delta T cells after 10 days in culture.

In some embodiments, the methods of the present invention are capable of generating/expanding a population of gamma delta T cells with greater efficiency as compared to prior art methods for generating/expanding gamma delta T cells.

As used herein, a reference prior art method for generating/expanding gamma delta T cells may be e.g. expansion in the presence of IL2 (in the absence of other added cytokines) and zoledonic acid. A reference prior art method for generating/expanding gamma delta T cells may be e.g. the method employed in Kobayashi and Tanaka Pharmaceuticals (Basel). 2015 March; 8(1): 40-61 or Deniger et al., Front Immunol. 2014; 5: 636, both of which are hereby incorporated by reference in in their entirety.

In some embodiments the methods yield a greater number of gamma delta T cells (i.e. generate/expand a larger population of gamma delta T cells) within a comparable period of time, and/or from a comparable starting population of immune cells (e.g. PBMCs, PBLs), as compared to prior art methods. In some embodiments, a method of the present invention results in the expansion of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the number of gamma delta T cells as compared to a reference prior art method.

In some embodiments the methods yield a population of immune cells having a greater proportion (i.e. comprising a higher percentage) of gamma delta T cells within a comparable period of time, and/or from a comparable starting population of immune cells (e.g. PBMCs, PBLs), as compared to prior art methods (prior to any step to further isolate/purify the generated/expanded gamma delta T cells). In some embodiments, a method of the present invention yields a population of immune cells (prior to any step to further isolate/purify the generated/expanded gamma delta T cells) comprising a percentage of gamma delta T cells which is one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the percentage of gamma delta T cells within a population of immune cells produced by a reference prior art method (prior to any step to further isolate/purify the generated/expanded gamma delta T cells).

In some embodiments the methods comprise a step of isolating or purifying the generated/expanded gamma delta T cells. Isolation/purification of the gamma delta T cells may be from the population of cells obtained following culture for the desired period of time. Essentially, isolation/purification of the gamma delta T cells involves separating the gamma delta T cells from other cells, e.g. other immune cells present in the culture at the end of the culture period. Various means for separating different kinds of immune cells are well known in the art, and include, e.g. cell sorting by Fluorescent-Activated Cell Sorting (FACS) or Magnetic-Activated Cell Sorting (MACS) based on expression of cell surface markers.

Gamma Delta T Cells

Gamma delta (γδ) T cells are a heterogeneous population of immune cells that consist of three major subtypes, Vδ1, Vδ2 and Vδ3, residing in specific anatomical sites. Gamma delta T cells and their biology is reviewed, for example, in Chien et al., Annu Rev Immunol. 2014; 32:121-55, which is hereby incorporated by reference in its entirety.

Certain methods disclosed herein are particularly applicable to gamma delta T cells of the Vδ2 subtype. In certain methods disclosed herein, the gamma delta T cells are Vγ9Vδ2 T cells. In some methods, the PBMCs have been obtained from a sample of peripheral blood and stored prior to use. That is, it is not necessary that the PBMCs are isolated from a blood sample and immediately cultured in a method according to the invention.

Methods disclosed herein may be used for the culture of ex vivo cells. Ex vivo cells have been taken from an individual. Methods disclosed herein may not involve the removal of cells from an individual, but may be applied to cells that have been previously obtained from that individual, such as cells in a sample obtained from that individual.

Preferably, gamma delta T cells produced by certain methods disclosed herein do not produce, or do not produce high levels of, IL17 and/or IL10. Preferably, they do not actively support tumor or T regulatory (Treg) cell growth. In some cases, a low or very low proportion of gamma delta T cells in the population of cells produced by the method produce IL17 and/or IL10. Preferably, fewer than 5% of the cells in the population, fewer than 4% of the cells in the population, fewer than 3% of the cells in the population, fewer than 2% of the cells in the population, or fewer than 1% of the cells in the population produce IL17 and/or IL10.

Methods disclosed herein may be used to generate gamma delta T cells useful for antigen presentation, and/or producing proinflammatory cytokines. Also disclosed are gamma delta T cells produced by these methods.

Gamma delta T cells disclosed herein may highly express antigen presentation markers, cell costimulation markers and/or effector markers. In this context “highly expressed” means at a level equal to, or preferably higher than, a gamma delta T cell generated in the presence of IL2 alone. “IL2 alone” refers to culture when IL2 is the only cytokine that has been added to the culture, or the only interleukin added to the culture. Certain gamma delta T cells disclosed herein express markers at 1.1, 1.2, 1.3, 1.4 or 1.5 times more than the expression of the same marker in a gamma delta T cell generated in the presence of 112 alone. Certain gamma delta T cells disclosed herein express markers at 2, 2.5, 3, or 3.5 times more than the expression of the same marker in a gamma delta T cell cultured in the generated of 112 alone.

Expression of markers may be determined by any suitable means. Expression may be gene expression or protein expression. Gene expression can be determined e.g. by detection of mRNA encoding the marker, for example by quantitative real-time PCR (qRT-PCR). Protein expression can be determined e.g. by detection of the marker, for example by antibody-based methods, for example by western blot, immunohistochemistry, immunocytochemistry, flow cytometry, or ELISA.

In preferred embodiments “expression” refers to protein expression of the relevant marker at/on the cell surface, and can be detected by flow cytometry using an appropriate marker-binding molecule.

Certain gamma delta T cells disclosed herein highly express one or more antigen presentation markers, such as HLA-ABC, and/or HLA-DR.

Certain gamma delta T cells disclosed herein highly express one or more cell costimulation markers, such as CD80, CD83, CD86, CD40 and/or ICAM-1.

These markers may be associated with presenting antigens to, and activating, CD4+ and CD8+ T cells.

Certain gamma delta T cells disclosed herein highly express one or more effector markers, such as CCR5, CCR6, CCR7, CD27 and/or NKG2D. These markers may be associated with homing of gamma delta T cells to lymph nodes, and interaction of gamma delta T cells with CD4+ and CD8+ T cells.

Certain gamma delta T cells disclosed herein express higher levels of ICAM-1 than a gamma delta T cell generated in the presence of IL2 alone. Such gamma delta T cells may have been generated in the presence of IL2 and another interleukin, such as IL7, IL15, IL18, IL21, or both IL18 and IL21. Such gamma delta T cells may be particularly useful where antigen presentation activity may be desirable.

Certain gamma delta T cells disclosed herein express higher levels of CD83 and/or CD80 than a gamma delta T cell generated in the presence of IL2 alone. Such gamma delta T cells may have been generated in the presence of IL2 and another interleukin, such as IL7, IL15 or IL18. Such gamma delta T cells may be particularly useful where antigen presentation activity may be desirable.

Certain gamma delta T cells disclosed herein express higher levels of CCR5, CCR7, CD27 and/or NKG2D than a gamma delta T cell generated in the presence of IL2. Such gamma delta T cells may be particularly useful where effector activity may be desirable. Gamma delta T cells disclosed herein may express at least 1.5, at least 2, at least 2.5 or at least 3 times more CCR5 than a gamma delta T cell generated in the presence of IL2.

Antigen Presentation Phenotypes

Gamma delta T cells may exhibit antigen presentation phenotypes. That is, gamma delta T cells may capture antigens and enable their recognition by other T cells, such as CD4+ and CD8+ T cells, including al T cells, thereby activating those T cells.

Gamma delta T cells generated/expanded according to the methods of the present invention may be employed as antigen-presenting cells in methods for expanding T cells having a desired specificity, e.g. virus-specific T cells.

Accordingly, the present invention provides a method for generating/expanding a population of antigen-specific T cells, comprising stimulating T cells by culture in the presence of gamma delta T cells generated/expanded according to the present invention, presenting a peptide of the antigen.

As used herein a “peptide” refers to a chain of two or more amino acid monomers linked by peptide bonds, which is 50 amino acids or fewer in length.

The antigen may be a peptide or polypeptide antigen. In some embodiments the antigen is associated with an infectious disease, an autoimmune disease, or a cancer. In some embodiments, the antigen is expressed by, or expression is upregulated in, a cell infected with an infectious agent (e.g. a virus or intracellular pathogen). In some embodiments, the antigen is expressed by, or expression is upregulated in, an autoimmune effector cell (e.g. an autoreactive T cell). In some embodiments, the antigen is expressed by, or expression is upregulated in, a cancer cell, e.g. a cell of a tumor. In some embodiments, the antigen is an antigen of an infectious agent (e.g. peptide/polypeptide of an infectious agent).

In connection with various aspects of the present invention, a cell (e.g. a gamma delta T cell) may present a peptide of an antigen as a consequence of infection by an infectious agent comprising/encoding the antigen/fragment thereof, uptake by the cell of the antigen/fragment thereof or expression of the antigen/fragment thereof. The presentation is typically in the context of an MHC molecule at the cell surface of the antigen-presenting cell.

It will be appreciated that reference to “a peptide” herein encompasses plural peptides. For example, cells presenting a peptide of an antigen may present plural peptides of the antigen. Methods for generating and/or expanding populations of e.g. antigen-specific T cells typically include several rounds of stimulation of T cells with antigen presenting cells presenting peptide of the antigen of interest (i.e. the virus for which the T cells are specific).

In one aspect, the present invention provides a method for generating or expanding a population of T cells specific for a virus, comprising stimulating T cells (e.g. within a population of immune cells, e.g. PBMCs, PBLs) by culture in the presence of gamma delta T cells expanded according to the methods described herein presenting a peptide of the virus.

The virus may be a dsDNA virus (e.g. adenovirus, herpesvirus, poxvirus), ssRNA virus (e.g. parvovirus), dsRNA virus (e.g. reovirus), (+)ssRNA virus (e.g. picornavirus, togavirus), (−)ssRNA virus (e.g. orthomyxovirus, rhabdovirus), ssRNA-RT virus (e.g. retrovirus) or dsDNA-RT virus (e.g. hepadnavirus). The present disclosure contemplates viruses of the families adenoviridae, herpesviridae, papillomaviridae, polyomaviridae, poxviridae, hepadnaviridae, parvoviridae, astroviridae, caliciviridae, picornaviridae, coronaviridae, flaviviridae, togaviridae, hepeviridae, retroviridae, orthomyxoviridae, arenaviridae, bunyaviridae, filoviridae, paramyxoviridae, rhabdoviridae and reoviridae. Viruses associated with a disease or disorder are of particular interest. Accordingly, the following viruses are contemplated: adenovirus, Herpes simplex type 1 virus, Herpes simplex type 2 virus, Varicella-zoster virus, Epstein-Barr virus, Human cytomegalovirus, Human herpesvirus type 8, Human papillomavirus, BK virus, JC virus, Smallpox, Hepatitis B virus, Parvovirus B19, Human Astrovirus, Norwalk virus, coxsackievirus, hepatitis A virus, poliovirus, rhinovirus, severe acute respiratory syndrome virus, hepatitis C virus, yellow fever virus, dengue virus, West Nile virus, TBE virus, Rubella virus, Hepatitis E virus, Human immunodeficiency virus, influenza virus, lassa virus, Crimean-Congo hemorrhagic fever virus, Hantaan virus, ebola virus, Marburg virus, measles virus, mumps virus, parainfluenza virus, respiratory syncytial virus, rabies virus, hepatitis D virus, rotavirus, orbivirus, coltivirus, and banna virus. In some embodiments, the virus is Epstein-Barr virus (EBV), human papillomavirus (HPV) or Hepatitis B Virus (HBV).

Accordingly, in some embodiments, the antigen is viral antigen. In some embodiments the antigen is, or is derived from, an EBV protein, which may be one of e.g. EBNA-1, EBNA-2, EBNA-3A, EBNA-3B, EBNA-3C, EBNA-LP, LMP-1, LMP-2A or LMP-2B. In some embodiments the antigen is, or is derived from, a HPV protein, which may be one of e.g. E1, E2, E3, E4, E5, E6, E7, L1, and/or L2. In some embodiments the antigen is, or is derived from, a HBV protein, which may be one of e.g. HBsAg, HBcAg, HBeAg, Hepatitis B virus DNA polymerase, HBx.

In aspects of the present invention wherein the gamma delta T cells generated/expanded according to the methods of the present invention are employed in methods to expand antigen-specific T cells, the gamma delta T cells may be treated in order that they express present one or more peptides of the relevant antigen. For example, the gamma delta T cells may be pulsed with peptides of the antigen according to methods well known to the skilled person. Antigenic peptides may be provided in a library of peptide mixtures (corresponding to one or more antigens), which may be referred to as pepmixes. Peptides of pepmixes may e.g. be overlapping peptides of 8-10 amino acids in length, and may cover all or part of the amino acid sequence of the relevant antigen(s).

Activation of CD4+ and CD8+ T cells involves IFNγ and TNFα. Gamma delta T cells produced by some of certain methods disclosed herein produce IFNγ and TNFα, and may therefore be useful for antigen presentation, and activation of CD4+ and/or CD4+ T cells.

In some cases, the population of cells generated by certain methods disclosed herein comprises at least 45%, at least 50%, at least 60%, or at least 65% cells that produce at least one of IFNγ and TNFα. Preferably, the population of cells generated by certain methods disclosed herein comprises at least 20%, at least 25%, at least 30%, at least 35%, at least 40%, at least 45%, or at least 50% gamma delta T cells that produce both IFNγ and TNFα.

Production of a given factor (e.g. IFNγ and TNFα) by gamma delta T cells can be measured by detecting gene or protein expression. Protein expression can be measured by various means known to those skilled in the art such as antibody-based methods, for example by ELISA, ELISPOT, western blot, immunohistochemistry, immunocytochemistry, flow cytometry or reporter-based methods. Production can also be determined by measuring levels of mRNA by quantitative real-time PCR (qRT-PCR), or by reporter-based methods.

In some cases, gamma delta T cells produced by certain methods disclosed herein are superior to monocyte-derived “classical Day 7” dendritic cells (DCs) in stimulating the proliferation of naïve CD4+ and/or CD8+ T cells. That is, in some embodiments the gamma delta T cells produced by methods disclosed herein stimulate proliferation of naïve CD4+ and/or CD8+ T cells to a greater extent than monocyte-derived “classical Day 7” DCs in a suitable assay. A suitable assay may involve stimulation of immune cells comprising naïve CD4+ and/or CD8+ T cells (e.g. a population of PBMCs) with gamma delta T cells produced by methods disclosed herein presenting a peptide of a viral antigen.

In some embodiments the gamma delta T cells produced by methods disclosed herein and employed as antigen presenting cells stimulate the proliferation of naïve CD4+ and/or CD8+ T cells to a greater extent than gamma delta T cells produced by a given reference prior art method.

Stimulation of proliferation of naïve CD4+ and/or CD8+ T cells “to a greater extent” may one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times.

Stimulation of cell proliferation can be determined by analysing cell division of stimulated cells over a period of time. Cell division for a given cell or population of cells can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. Proliferating cells may also be identified by analysis of incorporation of 5-ethyny-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety. Assays of antigen presentation function may involve treating (e.g. pulsing) the cells to be analysed with antigen/peptide thereof to be presented.

In some embodiments, the gamma delta T cells of the present invention are useful in methods for expanding T cell subsets of interest, e.g. in preference to other T cell subsets.

In some embodiments, gamma delta T cells produced by methods disclosed herein are superior at expanding antigen-specific T cells (e.g. antigen-specific CD8⁺ T cells). In some embodiments, gamma delta T cells produced by methods disclosed herein expand more antigen-specific T cells (e.g. antigen specific CD8⁺ T cells) as compared to the number of antigen-specific T cells expanded by monocyte-derived “classical Day 7” DCs, or gamma delta T cells produced by a given reference prior art method.

In some embodiments, gamma delta T cells produced by methods disclosed herein are useful as antigen presenting cells in methods for expanding T cells for generating a population of T cells with an increased proportion (i.e. a greater percentage) of antigen-specific T cells. An “increased proportion” of antigen-specific T cells may be e.g. one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the proportion of antigen-specific T cells within a population of T cells generated using e.g. monocyte-derived “classical Day 7” DCs, or gamma delta T cells produced by a given reference prior art method as antigen presenting cells.

In some embodiments, gamma delta T cells produced by methods disclosed herein expand fewer regulatory T cells (e.g. CD4+CD25+FOXP3 regulatory T cells) as compared to the number of regulatory T cells expanded by monocyte-derived “classical Day 7” DCs, or gamma delta T cells produced by a given reference prior art method.

In some embodiments, gamma delta T cells produced by methods disclosed herein are useful as antigen presenting cells in methods for expanding T cells for generating a population of T cells with a reduced proportion (i.e. a lower percentage) of regulatory T cells (e.g. CD4+CD25+FOXP3 regulatory T cells), which may be e.g. one of less than 1 times, less than 0.9 times, less than 0.8 times, less than 0.7 times, less than 0.6 times, less than 0.5 times, less than 0.4 times, less than 0.3 times, less than 0.2 times, or less than 0.1 times the proportion of regulatory T cells (e.g. CD4+CD25+FOXP3 regulatory T cells) within a population of T cells generated using e.g. monocyte-derived “classical Day 7” DCs, or gamma delta T cells produced by a given reference prior art method as antigen presenting cells.

In some embodiments, gamma delta T cells produced by methods disclosed herein expand fewer T cells having an exhausted phenotype as compared to the number of T cells having an exhausted phenotype expanded by monocyte-derived “classical Day 7” DCs, or gamma delta T cells produced by a given reference prior art method.

T-cell exhaustion is characterized by the stepwise and progressive loss of T-cell functions. Exhaustion is well-defined during chronic lymphocytic choriomeningitis virus (LCMV) infection and commonly develops under conditions of antigen-persistence, which occur following many chronic infections including hepatitis B virus, hepatitis C virus and human immunodeficiency virus infections, as well as during tumor metastasis. Exhaustion is not a uniformly disabled setting as a gradation of phenotypic and functional defects can manifest, and these cells are distinct from prototypic effector, memory and also anergic T cells. Exhausted T cells most commonly emerge during high-grade chronic infections, and the levels and duration of antigenic stimulation are critical determinants of the process. (Yi et al., Immunology April 2010; 129(4):474-481). Circulating human tumor-specific CD8+ T cells may be cytotoxic and produce cytokines in vivo, indicating that self- and tumor-specific human CD8+ T cells can reach functional competence after potent immunotherapy such as vaccination with peptide, incomplete Freund's adjuvant (IFA), and CpG or after adoptive transfer. In contrast to peripheral blood, T-cells infiltrating tumor sites are often functionally deficient, with abnormally low cytokine production and upregulation of the inhibitory receptors PD-1, CTLA-4, TIM-3 and LAG-3. Functional deficiency is reversible, since T-cells isolated from melanoma tissue can restore IFN-γ production after short-term in vitro culture. However, it remains to be determined whether this functional impairment involves further molecular pathways, possibly resembling T-cell exhaustion or anergy as defined in animal models. (Baitsch et al., J Clin Invest. 2011; 121(6):2350-2360).

As used herein a T cell having an exhausted phenotype may display surface expression of one or more of TIM-3, PD-1, CTLA-4 and LAG-3 (which can be determined e.g. by flow cytometry).

In some embodiments, gamma delta T cells produced by methods disclosed herein are useful as antigen presenting cells in methods for expanding T cells for generating a population of T cells with a reduced proportion (i.e. a lower percentage) of T cells having an exhausted phenotype (e.g. TIM-3⁺, PD-1⁺, CTLA-4⁺, and/or LAG-3⁺ T cells). The reduced proportion may be e.g. one of less than 1 times, less than 0.9 times, less than 0.8 times, less than 0.7 times, less than 0.6 times, less than 0.5 times, less than 0.4 times, less than 0.3 times, less than 0.2 times, or less than 0.1 times the proportion of T cells having an exhausted phenotype within a population of T cells generated using e.g. monocyte-derived “classical Day 7” DCs, or gamma delta T cells produced by a given reference prior art method as antigen presenting cells.

In some embodiments, gamma delta T cells produced by methods disclosed herein employed as antigen presenting cells expand T cells (e.g. CD8⁺ T cells, e.g. CTLs) having improved effector function as compared to T cells expanded by monocyte-derived “classical Day 7” DCs, or gamma delta T cells produced by a given reference prior art method.

In some embodiments effector function may be e.g. cell lysis of a target cell expressing the antigen for which the T cell is specific and/or expression of one or more of granzyme A, granzyme B, granulysin, perforin, IFNγ, TNFα and IL-17A.

In some embodiments, gamma delta T cells produced by methods disclosed herein are useful as antigen presenting cells in methods for expanding T cells for generating T cells (e.g. CD8⁺ T cells, e.g. CTLs) having improved effector function as compared to the effector function displayed by T cells generated using e.g. monocyte-derived “classical Day 7” DCs, or gamma delta T cells produced by a given reference prior art method as antigen presenting cells. “improved effector function” may be a level of the relevant function (e.g. a level of cell lysis, or a level of expression of the relevant factor) which is e.g. one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the level of the relevant function displayed by T cells (e.g. CD8⁺ T cells, e.g. CTLs) generated using e.g. monocyte-derived “classical Day 7” DCs, or gamma delta T cells produced by a given reference prior art method as antigen presenting cells.

Effector Phenotypes

Gamma delta T cells may exhibit cytolytic phenotypes. That is, they may target and/or lyse tumor cells.

Gamma delta T cells produced by certain methods disclosed herein may produce granzyme A, granzyme B, perforin and/or granulysin. The gamma delta T cells may be able to target and/or lyse tumor cells. The gamma delta T cells may be useful for targeting and/or lysing viral antigen expressing tumor cells, such as EBV expressing tumor cells.

Gamma delta T cells produced by certain methods disclosed herein may exhibit antigen presentation phenotypes, effector phenotypes, or both antigen presentation and effector phenotypes.

Cytolytic properties of the gamma delta T cells such as tumor cell lysis and/or production of granzyme A, granzyme B, perforin and/or granulysin may be dependent on ligation of NKG2D by its ligand.

In some embodiments, gamma delta T cells generated/expanded by the methods disclosed herein display increased expression of one or more factors as compared to the level of expression by gamma delta T cells generated/expanded by a reference prior art method (e.g. following stimulation with a given cell type, e.g. a cancer cell or C666-1, Hep3B, DLD-1 or K562 cells).

In some embodiments a factor may be selected from granzyme A, granzyme B, granulysin, perforin, IFNγ, IL-17A, IL-8, Eotaxin, IP-10, MIG, GRO A, MIUP-3A, I-TAC, MCP-1, RANTES, MIP-1A, MIP-1B and ENA-78.

In some embodiments “increased expression” is one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the level of expression by the gamma delta T cells generated/expanded by a reference prior art method.

Expression of factors may be determined by any suitable means. Expression may be gene expression or protein expression. Gene expression can be determined e.g. by detection of mRNA encoding the factor, for example by quantitative real-time PCR (qRT-PCR). Protein expression can be determined e.g. by detection of the factor, for example by antibody-based methods, for example by western blot, immunohistochemistry, immunocytochemistry, flow cytometry, or ELISA.

In some embodiments, gamma delta T cells generated/expanded by the methods disclosed herein display increased lysis of target cells (e.g. cancer cells, e.g. C666-1, Hep3B, DLD-1 or K562 cells) as compared to the level of lysis displayed by gamma delta T cells generated/expanded by a reference prior art method. For example, gamma delta T cells generated/expanded by the methods disclosed herein may cause cell lysis of a greater proportion (e.g. a higher percentage) of a target cell population in an appropriate assay of such activity, as compared to gamma delta T cells generated/expanded by a reference prior art method.

In some embodiments “increased lysis” is one of more than 1 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.1 times, more than 2.2 times, more than 2.3 times, more than 2.4 times, more than 2.5 times, more than 2.6 times, more than 2.7 times, more than 2.8 times, more than 2.9 times, more than 3 times, more than 3.1 times, more than 3.2 times, more than 3.3 times, more than 3.4 times, more than 3.5 times, more than 3.6 times, more than 3.7 times, more than 3.8 times, more than 3.9 times, more than 4 times, more than 4.1 times, more than 4.2 times, more than 4.3 times, more than 4.4 times, more than 4.5 times, more than 4.6 times, more than 4.7 times, more than 4.8 times, more than 4.9 times, or more than 5 times the level of lysis displayed by the gamma delta T cells generated/expanded by a reference prior art method, in a comparable assay.

Cell lysis by gamma delta T cells can be investigated, for example, using any of the methods reviewed in Zaritskaya et al., Expert Rev Vaccines (2011), 9(6):601-616, hereby incorporated by reference in its entirety. One example of an assay for cytotoxicity of a T cell for to a target cell is the ⁵¹Cr release assay, in which target cells are treated with ⁵¹Cr, which they internalise. Lysis of the target cells results in the release of the radioactive ⁵¹Cr into the cell culture supernatant, which can be detected.

Interleukins

Methods disclosed herein relate to the culture of PBMCs in the presence of one or more interleukins. Certain methods may involve culture in the presence of exogenous interleukin. That is, interleukin that has been added to the culture, such as added to the culture media. The interleukins employed in the methods of the present invention may be recombinantly produced, and/or obtained from a suitable source for clinical application.

In accordance with various aspects disclosed herein, where culture is performed in the “presence of” a given cytokine, the relevant cytokine (e.g. recombinant and/or exogenous cytokine) may have been added to the culture. Where culture is performed in the “absence of” a given cytokine, the relevant cytokine (e.g. recombinant and/or exogenous cytokine) will not have been added to the culture.

In some cases, the cells are cultured in media that has been supplemented with the one or more interleukins. In others, the media comprises the one or more interleukins. Some of certain methods involve culturing PBMCs in the presence of two or more interleukins simultaneously. That is, the culture comprises a plurality of interleukins, rather than sequential culture of the cells in each different cytokine individually. However, having cultured the cells in one particular interleukin, or combination of interleukins, the cells may be subsequently transferred to a further culture using a different interleukin or combination of interleukins.

IL2 has been used for generating gamma delta T cells for the clinic. In some methods disclosed herein, gamma delta T cells may be generated in the presence of at least 150 IU/ml, at least 160 IU/ml, at least 170 IU/ml, at least 180 IU/ml, at least 190 IU/ml, at least 200 IU/ml of IL2. Preferably, the gamma delta T cells are generated in the presence of 200 IU/ml IL2.

In some embodiments IL2 is added to the culture at a final concentration 50-500 IU/ml, 50-400 IU/ml, 50-300 IU/ml, 50-250 IU/ml, 50-200 IU/ml, 75-500 IU/ml, 75-400 IU/ml, 75-300 IU/ml, 75-250 IU/ml, 75-200 IU/ml, 100-500 IU/ml, 100-400 IU/ml, 100-300 IU/ml, 100-250 IU/ml, 100-200 IU/ml, 125-500 IU/ml, 125-400 IU/ml, 125-300 IU/ml, 125-250 IU/ml, 125-200 IU/ml, 150-500 IU/ml, 150-400 IU/ml, 150-300 IU/ml, 150-250 IU/ml, or 150-200 IU/ml.

As used herein, IU means International Unit, and is a measure of activity determined by an International Standard. The International Standard for 112 is NIBSC 86/504.

In some methods disclosed herein, IL2 may be used in combination with other cytokines. In particular, IL2 may be used in combination with IL21.

In some methods disclosed herein, gamma delta T cells may be generated in the presence of Interleukin 15 (IL15) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL15.

In some embodiments IL15 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml. In some methods disclosed herein, IL15 may be used alone or in combination with other cytokines. For example, IL15 may be used in combination with 1121, or IL21 and IL18.

In some methods disclosed herein, gamma delta T cells may be generated in the presence of Interleukin 21 (IL21) at a concentration of at least 15 ng/ml, at least 20 ng/ml, at least 25 ng/ml, at least 5 ng/ml, at least 26 ng/ml, at least 27 ng/ml, at least 28 ng/ml, at least 29 ng/ml or at least 30 ng/ml. Preferably, certain methods disclosed herein involve culture of gamma delta T cells in the presence of 30 ng/ml of IL21.

In some embodiments IL15 is added to the culture at a final concentration 5-80 ng/ml, 5-70 ng/ml, 5-60 ng/ml, 5-50 ng/ml, 5-40 ng/ml, 5-30 ng/ml, 10-80 ng/ml, 10-70 ng/ml, 10-60 ng/ml, 10-50 ng/ml, 10-40 ng/ml, 10-30 ng/ml, 15-80 ng/ml, 15-70 ng/ml, 15-60 ng/ml, 15-50 ng/ml, 15-40 ng/ml, 15-30 ng/ml, 20-80 ng/ml, 20-70 ng/ml, 20-60 ng/ml, 20-50 ng/ml, 20-40 ng/ml, 20-30 ng/ml, 25-80 ng/ml, 25-70 ng/ml, 25-60 ng/ml, 25-50 ng/ml, 25-40 ng/ml, or 25-30 ng/ml.

In some methods disclosed herein, IL121 may be used alone or in combination with other cytokines. For example, IL21 may be used in combination with 11L2 or IL15. IL21 may be used in combination with IL18, and 112 or IL15.

In some methods disclosed herein, gamma delta T cells are generated in the presence of Interleukin 7 (IL7) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL7.

In some embodiments IL7 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml.

In some methods disclosed herein, gamma delta T cells may be generated in the presence of Interleukin 18 (IL18) at a concentration of at least 2 ng/ml, at least 3 ng/ml, at least 4 ng/ml, at least 5 ng/ml, at least 6 ng/ml, at least 7 ng/ml, at least 8 ng/ml, at least 9 ng/ml or at least 10 ng/ml. Preferably, certain methods disclosed herein involve culture of gamma delta T cells in the presence of 10 ng/ml of IL18.

In some embodiments IL18 is added to the culture at a final concentration 1-30 ng/ml, 1-25 ng/ml, 1-20 ng/ml, 1-15 ng/ml, 1-10 ng/ml, 2-30 ng/ml, 2-25 ng/ml, 2-20 ng/ml, 2-15 ng/ml, 2-10 ng/ml, 3-30 ng/ml, 3-25 ng/ml, 3-20 ng/ml, 3-15 ng/ml, 3-10 ng/ml, 4-30 ng/ml, 4-25 ng/ml, 4-20 ng/ml, 4-15 ng/ml, 4-10 ng/ml, 5-30 ng/ml, 5-25 ng/ml, 5-20 ng/ml, 5-15 ng/ml, or 5-10 ng/ml.

Methods disclosed herein relate to the culture of gamma delta T cells in the presence of one or more Interleukin. In particular, methods disclosed herein relate to culture of gamma delta T cells in the presence of:
IL2 and IL21;
IL15;
IL21;
IL15 and IL21;
IL2 and IL18;
IL15, IL18 and IL21.
IL2 and IL7;
IL2 and IL15;
IL2, IL18 and IL21;
IL15 and IL7; or
IL15 and IL18.

Certain methods disclosed herein relate to culture of gamma delta T cells in the presence of IL15. In particular, methods disclosed herein relate to the culture of gamma delta T cells in the presence of IL15 and IL21. In some cases, the gamma delta T cells are generated in the presence of IL15 and IL21 and IL18.

Certain methods disclosed herein relate to culture of gamma delta T cells in the presence of IL21. In particular, methods disclosed herein relate to the culture of gamma delta T cells in the presence of IL21 and IL2, or IL21 and IL15. In some cases, the gamma delta T cells are generated in the presence of IL21 and IL2 and IL18. In some cases, the gamma delta T cells are generated in the presence of IL21 and IL15 and IL18.

In the methods of the present disclosure, the one or more interleukins are added to the culture on one or more of days 1, 2, 3, 4, 5, 6, 7, 8, 9 and 10. In some embodiments the interleukins are added to the culture at the same time as, or after, the addition of an agent capable of stimulating the proliferation of gamma delta T cells (e.g. zoledronic acid). In some embodiments the interleukins are added on day 1 of the culture. In some embodiments the interleukins are added to the culture on day 3 of the culture. In some embodiments the interleukins are added to the culture on days 1 and 3 of the culture. In some embodiments the interleukins are added to the culture: daily, every 2 days, every 3 days, every 4 days or every 5 days.

In some embodiments the agent capable of stimulating the proliferation of gamma delta T cells is added at the same time as adding one or more interleukins to the culture.

T Cell Medium

Methods disclosed herein relate to the culture of gamma delta T cells in cell culture medium, and particularly in T cell medium. T cell medium is a liquid containing nutrients that supports the growth of T cells, such as amino acids, inorganic salts, vitamins, and sugars. As used here, the term T cell medium refers to medium that does not contain cytokines, such that the amount of cytokine in the culture may be manipulated through the addition of one or more cytokines. In some cases, the T cell medium does not contain interleukins, such that the amount of interleukin in the culture may be manipulated through the addition of one or more interleukins.

Suitable T cell medium includes Click's medium, or OpTimizer® (CTS®), medium. Stemline® T cell expansion medium (Sigma-Aldrich), AIM V® medium (CTS®), TexMACS® medium (Miltenyi Biotech), ImmunoCult® medium (Stem Cell Technologies), PRIME-XV® T-Cell Expansion XSFM (Irvine Scientific), Iscoves medium and RPMI-1640 medium.

In particular, certain methods disclosed herein relate to the culture of gamma delta T cells in Clicks medium, or OpTimizer® medium.

In preferred aspects, certain methods disclosed herein relate to culture in OpTimizer® T cell medium (CTS®).

Medium used in the present invention may be serum free medium, or may comprise serum. In some methods, serum may be added to serum free medium.

In some embodiments the medium may comprise one or more cell culture medium additives. Cell culture medium additives are well known to the skilled person, and include antibiotics (e.g. penicillin, streptomycin), serum, L-glutamine, growth factors, etc.

Serum

Culture medium is commonly supplemented with serum in cell culture methods. Serum may provide factors required for cell attachment, grown and proliferation, and thus may act as a growth supplement.

Serum may be serum of human or animal origin. The serum may be human serum. Serum may be pooled human AB serum, FBS (Fetal Bovine Serum) or defined FBS. The serum may be autologous serum.

Preferably, the serum is a clinically acceptable serum. The serum may be sterile-filtered. The serum may be heat-inactivated.

Some methods disclosed herein relate to the culture of gamma delta T cells in culture medium supplemented with 1%, 2%, 3%, 4%, 5%, 6%, 7%, 8%, 9%, 10%, 11%, 12%, 13%, 14% or 15% serum. In some cases, the culture medium may be supplemented with at least 1% serum, at least 2% serum, at least 3% serum, at least 4% serum, at least 5% serum, at least 6% serum, at least 7% serum, at least 8% serum, at least 9% serum, at least 10% serum, at least 11% serum, at least 12% serum, at least 13% serum, at least 14% serum, at least 15% serum.

In some methods, the culture medium may be supplemented with 10% serum, or at least 10% serum.

In some cases, the culture medium may be supplemented with less than 30% serum, less than 25% serum, less than 20% serum, or less than 15% serum.

In some cases, the culture medium may be supplemented with one of 1-20%, 1-15% or 1-10% serum. In some cases, the culture medium may be supplemented with one of 1-10%, 1-8% or 1-5% serum.

Compositions

The invention described herein also provides compositions comprising gamma delta T cells produced according the methods described herein.

The gamma delta T cells may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the gamma delta T cells in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In particular embodiments the compositions may be formulated for intratumoral or intravenous administration.

In accordance with the invention described herein methods are also provided for the production of pharmaceutically useful compositions, such methods of production may comprise one or more steps selected from: isolating/purifying gamma delta T cells produced according to the methods described herein; and/or mixing gamma delta T cells produced according to the methods described herein with a pharmaceutically acceptable carrier, adjuvant, excipient or diluent.

For example, a further aspect the invention described herein relates to a method of formulating or producing a medicament or pharmaceutical composition, comprising formulating a pharmaceutical composition or medicament by mixing gamma delta T cells produced according to the methods described herein with a pharmaceutically acceptable carrier, adjuvant, excipient or diluent.

Uses of, and Methods Using, the Gamma Delta T Cells and Compositions

The gamma delta T cells and pharmaceutical compositions according to the present invention find use in therapeutic and prophylactic methods.

The present invention provides a gamma delta T cell or pharmaceutical composition according to the present invention for use in a method of medical treatment or prophylaxis.

The present invention also provides the use of a gamma delta T cell or pharmaceutical composition according to the present invention in the manufacture of a medicament for treating or preventing a disease or disorder.

The present invention also provides a method of treating or preventing a disease or disorder, comprising administering to a subject a therapeutically or prophylactically effective amount of a gamma delta T cell or pharmaceutical composition according to the present invention.

The disease or disorder to be treated/prevented may be any disease/disorder which would derive therapeutic or prophylactic benefit from an increase in the number of gamma delta T cells.

Also as described herein, the methods of the present invention are useful for generating/expanding gamma delta T cells which are in turn useful as antigen presenting cells for use in methods for expanding antigen-specific T cells, e.g. virus-specific T cells useful in methods for treating/preventing diseases/disorders (e.g. viral disease and virus-associated cancers).

In particular embodiments, the disease or disorder to be treated/prevented may be a cancer. In some embodiments, the gamma delta T cells and compositions of the present invention are capable of treating or preventing a cancer (e.g. inhibit the development/progression of the cancer, delay/prevent onset of the cancer, reduce/delay/prevent tumor growth, reduce/delay/prevent metastasis, reduce the severity of the symptoms of the cancer, reduce the number of cancer cells, reduce tumour size/volume, and/or increase survival (e.g. progression free survival)).

Administration

Administration of a gamma delta T cell or pharmaceutical composition according to the invention is preferably in a “therapeutically effective” or “prophylactically effective” amount, this being sufficient to show benefit to the subject.

The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of gamma delta T cells or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments gamma delta T cells or pharmaceutical compositions of the present invention may be administered alone or in combination with one or more other agents, either simultaneously or sequentially dependent upon the condition to be treated/prevented.

In some embodiments gamma delta T cells or pharmaceutical compositions disclosed herein may be administered in combination with an agent capable of activating gamma delta T cells e.g. an agent comprising a phospho antigen and/or aminobisphosphonate. In some embodiments the agent may be pamidronate or zoledronic acid.

Simultaneous administration refers to administration of the gamma delta T cells/pharmaceutical composition and agent together, for example as a pharmaceutical composition containing both of (i) the gamma delta T cells/pharmaceutical composition and (ii) the agent, in combined preparation or immediately after each other and optionally via the same route of administration, e.g. to the same artery, vein or other blood vessel.

Sequential administration refers to administration of one or other of the (i) gamma delta T cells/pharmaceutical composition and (ii) the agent after a given time interval by separate administration. It is not required that the two agents are administered by the same route, although this is the case in some embodiments. The time interval may be any time interval.

In some embodiments, the methods of the present invention comprise additional therapeutic or prophylactic intervention for the treatment or prevention of a disease or disorder, e.g. chemotherapy, immunotherapy, radiotherapy, surgery, vaccination and/or hormone therapy.

Chemotherapy and radiotherapy respectively refer to treatment of a cancer with a drug or with ionising radiation (e.g. radiotherapy using X-rays or γ-rays).

The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A therapeutic or prophylactic intervention may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1 BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitoi, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, AnandronZ, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXARS, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte-Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mybcel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortefe, Solu-Medrol, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon, Xeloda®, Zanosar®, Zevalin™, Zinecard®, Zoladex®, Zoledronic acid, Zolinza, Zometa®.

Cancer

In some embodiments, the disease or disorder to be treated or prevented in accordance with various aspects of the present disclosure is a cancer. The cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

Gamma delta T cells produced by certain methods disclosed herein may be useful in the treatment of leukemia, nasopharyngeal carcinoma, breast carcinoma, hepatocellular carcinoma, lung carcinoma, renal cell carcinoma, pancreatic adenocarcinoma, prostate carcinoma, or neuroblastoma.

Gamma delta T cells produced by certain methods disclosed herein may be useful for the treatment or inhibition of viral-related cancers, such as EBV related/associated cancer, or an HPV associated cancer.

“EBV associated” and “HPV associated” cancers may be a cancers which are caused or exacerbated by infection with the respective viruses, cancers for which infection is a risk factor and/or cancers for which infection is positively associated with onset, development, progression, severity or metastasis.

EBV-associated cancers which may be treated with cells produced by methods of the disclosure include nasopharyngeal carcinoma (NPC) and gastric carcinoma (GC).

HPV-associated medical conditions that may be treated with cells produced by methods of the disclosure include at least dysplasias of the genital area(s), cervical intraepithelial neoplasia, vulvar intraepithelial neoplasia, penile intraepithelial neoplasia, anal intraepithelial neoplasia, cervical cancer, anal cancer, vulvar cancer, vaginal cancer, penile cancer, genital cancers, oral papillomas, oropharyngeal cancer.

In some embodiments, the cancer to be treated in accordance with various aspects of the present disclosure is one or more of nasopharyngeal carcinoma (NPC; e.g. Epstein-Barr Virus (EBV)-positive NPC), cervical carcinoma (CC; e.g. human papillomavirus (HPV)-positive CC), oropharyngeal carcinoma (OPC; e.g. HPV-positive OPC), gastric carcinoma (GC; e.g. EBV-positive GC), hepatocellular carcinoma (HCC; e.g. Hepatitis B Virus (HBV)-positive HCC), lung cancer (e.g. non-small cell lung cancer (NSCLC)) and head and neck cancer (e.g. cancer originating from tissues of the lip, mouth, nose, sinuses, pharynx or larynx, e.g. head and neck squamous cell carcinoma (HNSCC)).

Adoptive Transfer

Gamma delta T cells produced by some methods disclosed herein may be useful for adoptive T cell therapy. Adoptive cell therapy involves the introduction of cells into a patient in need of treatment. In some cases, the cells are derived from the patient that they are introduced to (autologous cell therapy). That is, cells may have been obtained from the patient, generated according to methods described herein, and then returned to the same patient. Methods disclosed herein may also be used in allogenic cell therapy, in which cells obtained from a different individual are introduced into the patient.

Accordingly, the present invention provides a method of treatment or prophylaxis comprising adoptive transfer of gamma delta T cells produced (i.e. generated or expanded) according to the methods of the present invention. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of gamma delta T cells is reviewed, for example, in Kobayashi and Tanaka Pharmaceuticals (Basel). 2015 March; 8(1): 40-61, and Deniger et al., Front Immunol. 2014; 5: 636, incorporated by reference herein.

In the present invention, adoptive transfer is performed with the aim of introducing, or increasing the frequency of, gamma delta T cells in a subject.

Accordingly, the present invention provides a method of treating or preventing a disease or disorder in a subject, comprising:
(a) isolating PBMCs from a subject;
(b) generating or expanding a population of gamma delta T cells according to the method of the present invention, and;
(c) administering the gamma delta T cells to a subject.

In some embodiments, the subject from which the PBMCs are isolated is the subject administered with the gamma delta T cells (i.e., adoptive transfer is of autologous cells). In some embodiments, the subject from which the PBMCs are isolated is a different subject to the subject to which the gamma delta T cells are administered (i.e., adoptive transfer is of allogenic cells).

In some embodiments the method may comprise one or more of the following steps: taking a blood sample from a subject; isolating PBMCs from the blood sample; generating or expanding gamma delta T cells as described herein; collecting the gamma delta T cells; mixing the gamma delta T cells with an adjuvant, diluent, or carrier; administering the gamma delta T cells or composition to a subject.

The skilled person is able to determine appropriate reagents and procedures for adoptive transfer of gamma delta T cells generated or expanded according to the methods of the present invention for example by reference to Nakajima et al., Eur J Cardiothorac Surg. 2010; 37:1191-7 and Abe et al., Exp Hematol. 2009; 37:956-68, both of which are hereby incorporated by reference in their entirety.

As explained herein, gamma delta T cells obtained by methods according to the present invention are also useful in methods for expanding antigen-specific T cells, and antigen-specific T cells expanded according to such methods are provided with certain advantageous properties making them particularly suited to use in methods of treating/preventing diseases/disorders.

Adoptive transfer of T cells is described, for example, in Kalos and June 2013, Immunity 39(1): 49-60, which is hereby incorporated by reference in its entirety.

Accordingly, the present invention provides a method of treating or preventing a disease or disorder in a subject, comprising:
(a) isolating PBMCs from a subject;
(b) generating or expanding a population of gamma delta T cells according to the method of the present invention;
(c) generating or expanding a population of antigen-specific T cells by a method comprising stimulating T cells by culture in the presence of gamma delta T cells generated/expanded according to (b) presenting a peptide of the antigen; and
(d) administering the antigen-specific T cells to a subject.

It will be clear to the person skilled in the art that the therapeutic utility extends to essentially any disease/condition which would benefit from a reduction in the number of cells comprising/expressing the relevant antigen.

Subjects

The subject to be treated with the gamma delta T cells or pharmaceutical compositions of the invention may be any animal or human. The subject is preferably mammalian, more preferably human. The subject may be a non-human mammal, but is more preferably human. The subject may be male or female. The subject may be a patient. A subject may have been diagnosed with a disease or disorder requiring treatment, may be suspected of having such a disease or disorder (e.g. a cancer), or may be at risk from developing such a disease or disorder.

In embodiments according to the present invention the subject is preferably a human subject. In some embodiments, the subject to be treated according to a therapeutic or prophylactic method of the invention herein is a subject having, or at risk of developing, a cancer. In embodiments according to the present invention, a subject may be selected for treatment according to the methods based on characterisation for certain markers of such disease/disorder. A subject may have been diagnosed with the disease or disorder requiring treatment, or be suspected of having such a disease or disorder.

### BRIEF DESCRIPTION OF THE FIGURES

Embodiments and experiments illustrating the principles of the invention will now be discussed with reference to the accompanying figures in which:

FIGS. 1A to 1D. Bar charts showing evaluation of different medium, serum and cytokine combinations on cell proliferation and purity. (1A) effect of medium and serum combination on cell proliferation and (1C) purity. (1B) effect of interleukin supplementation on cell proliferation and (1D) purity for PBMCs cultured in OpTimizer media with 10% defined FBS supplementation.

FIGS. 2A and 2B. Bar charts and histograms showing antigen presentation and effector phenotypic markers exhibited by gamma delta T cells. (2A) gamma delta T cells generated in the presence of IL15 and IL21 highly expressed antigen presentation markers (HLA-ABC and HLA-DR), T cell costimulation markers (i.e. CD80, CD83, CD40 and ICAM-1) as well as effector markers (i.e. CCR5, CCR6, CCF7, CD27 and NKG2D), and this phenotypic profile was representative of all the gamma delta T cells produced under different cytokine combinations tested (2B) indicating that they had the potential to perform multiple functions of antigen presentation, T cell constimulation and direct tumor cell lysis.

FIGS. 3A to 3I. Histograms, graphs, bar charts and heatmaps showing response of gamma delta T cells to tumor cells (3A) expression of ligands amongst four tumor cell lines (dotted open and solid shaded histograms represent isotype control and tests respectively) (3B, 3C, 3G) percentage lysis of tumor cells by gamma delta T cells in 2 hour assay. (3D, 3E, 3F, 3I) evaluation of mode of direct tumor cytolysis by gamma delta T cells through cluster analysis of secreted granzymes A and B, granulysin, perforin, IFN-γ and proinflammatory chemokines. (3H) cytolytic activity of gamma delta T cells against C666-1 tumor cells in the presence or absence of anti-NKG2D blocking antibody, as measured by granzyme A production.

FIGS. 4A to 4E. Histograms, bar charts and graphs showing ex vivo generated gamma delta T cells were more efficient than monocyte-derived dendritic cells in stimulating the proliferation of naïve CD4+ and CD8+ T cells Gamma delta T cells pulsed with peptides derived from either EBV or NY-ESO1 and cocultured with CFSE-labelled naïve CD4+ and CD8+ T cells for two weeks. (4A, 4B, 4C) proliferation by naïve CD4+ and CD8+ T cells. In the histograms, each peak represents a round of T cell proliferation. The percentage of proliferating cells is shown. (40, 4E) percentage of EBV- and NY-ESO-1-specific CD8⁺ T cells detected following simulation of naïve T cells with peptide-pulsed γδ T cells generated under different culture conditions, as compared to stimulation with peptide-pulsed monocyte-derived DCs.

FIGS. 5A to 5C. Bar charts, graphs, histograms and pie charts showing gamma delta T cells pulsed with EBV-LMP2A overlapping pooled peptides stimulated fewer CD4+CD25+FOXP3+ Tregs, and fewer exhausted CD4+ and CD8+ T cells from PBLs as compared to peptide-pulsed monocyte-derived dendritic cells (5A) percentage of CD3+ T lymphocytes, CD4+ T cells, CD8+ T cells and Tregs in coculture following 2 weeks of coculture of the peptide-pulsed cells with PBLs. (5B) expression of exhaustion markers PD-1, TIM-3, LAG-3, CTLA-4 and activation marker CD28 on CD8+ T cells, CD4+ T cells, gamma-delta T cells and Tregs after 2 weeks of coculture. (5C) secretion of IFN-gamma, TNF-alpha and IL-17 by CD8+ and CD4+ T cells in response to stimulation with EBV-LMP2A overlapping pooled peptides.

FIGS. 6A to 6D. Schematic, photographs and table showing the procedures and results of an In vivo experiment In mice Investigating anti-tumor effects for administration of γδ T cells (6A) schematic representation of the procedures for Experiment 1. (6B) photographs of the tumors harvested from mice at the end of the experiment. (6C) photographs of spleens harvested from mice at the end of the experiment. (6D) Table summarising measurements of the tumors harvested from mice at the end of the experiment.

FIGS. 7A and 7B. Schematic and table showing the procedures and results of an In vivo experiment in mice Investigating anti-tumor effects for administration of γδ T cells (7A) schematic representation of the procedures for Experiment 2. (7B) Table summarising measurements of the tumors harvested from mice at the end of the experiment.

FIGS. 8A and 8B. Schematic and table showing the procedures and treatments for an In vivo experiment In mice Investigating anti-tumor effects for administration of γδ T cells and zoledronic acid (6A) schematic representation of the procedures for Experiment 3. (6B) Table summarising the treatments for each of the five treatment groups of Experiment 3.

### EXAMPLES

### Example 1: Materials and Methods

Ethics Approval and Participant Consent

All healthy donors and nasopharyngeal cancer patients are confirmed to have given written informed consent to a tissue and blood procurement study allowing ex vivo experimentation, which is approved by the National Cancer Centre Singapore's Office of Regulatory Affairs, Institutional Review Board (IRB).

Antibodies

The following monoclonal human antibodies (MoAbs) were used: (1) γδTCR [FITC-conjugated, clone Immu360; Beckman Coulter, Indianapolis, USA]; (2) γδTCR [PE-conjugated, clone 11F2; BD Bioscience, New Jersey, USA]; (3) CD3 (Pacific Blue-conjugated, clone UCHT1, mouse IgG1κ; BD Pharminen, New Jersey, USA); (4) HLA-ABC (APC-Cy7-conjugated, clone W6/32, mouse IgG2aκ; Biolegend); (5) HLA-DR (FITC-conjugated, clone L243, mouse IgG2a; BD Bioscience); (6) CD40 (PE-Cy7-conjugated, clone 5C3, mouse IgG1κ; BD Pharminen); (7) CD80 (PE-Cy7-conjugated, clone L307.4, mouse IgG1κ; BD Pharminen); (8) CD83 (PE-Cy7-conjugated, clone HB15e, mouse IgG1κ; BD Pharminen); (9) CD86 (PE-Cy7-conjugated, clone 2331/FUN-1, mouse IgG1κ; BD Pharminen); (10) CD54 (ICAM-1) [APC-conjugated, clone HA58, mouse IgG1κ; BD Pharminen); (11) ICOSL (FITC-conjugated, clone MIH12, mouse IgG1κ; Miltenyi Biotec GmbH, Bergisch Gladbach, Germany); (12) CD1d (APC-conjugated, clone CD1d42, mouse IgG1κ; BD Pharminen); (13) CCR5 (CD195) [APC-Cy7-conjugated, clone 2D7/CCR5, mouse IgG2aκ; BD Pharminen]; (14) CCR6 (CD196) [FITC-conjugated, clone G034E3, mouse IgG2bκ; Biolegend]; (15) CCR7 (CD197) [APC-conjugated, clone G043H7, mouse IgG2aκ; Biolegend]; (16) CD27 (FITC-conjugated, clone M-T271, mouse IgG1κ; BD Pharminen); (17) NKG2D (CD314) [APC-conjugated, clone 1D11, mouse IgG1κ; BD Pharminen]; (18) PD-1 (CD279) [APC-conjugated, clone MIH4, mouse IgG1κ; eBioscience, San Diego, USA); (19) CLTA-4 (PE-Cy7-conjugated, clone 14D3, mouse IgG2aκ; eBioscience); (20) Fas ligand (FASL/CD178) [APC-conjugated, clone NOK-1, mouse IgG1κ; BD Pharminen]; (21) IFN-γ (PE-conjugated, clone 4S.B3, mouse IgG1κ; Biolegend, San Diego, USA); (22) TNF-α (APC-Cy7-conjugated, clone Mab11, mouse IgG1κ; Biolegend); (23) IL-10 (PE-Cy7-conjugated, clone JES3-9D7, mouse IgG1κ; Biolegend); (24) IL-17A (APC-conjugated, clone BL168, mouse IgG1K; Biolegend); (25) TIM3 (CD366, PE-conjugated, clone F38-2E2 Mouse IgG IgG1κ, eBioscience); (26) LAG3 (CD223, PE-Cy7-conjugated, clone 3DS223H, mouse IgG1κ, eBioscience; (27) MICA (PE-conjugated, clone #159227, mouse IgG2b; R&D Systems); (28) MICB (APC-conjugated, clone #236511, mouse IgG2b; R&D Systems); (29) BTN3A1 (CD277) [APC-conjugated, clone BL168, mouse IgG1; Novus Biologicals, Colorado, USA); (30) NKG2D blocking antibody (purified, clone 1D11, mouse IgG1κ; Biolegend); (31) γδ TCR blocking antibody (purified, clone B1, mouse IgG1κ; Biolegend). Cells were washed twice with DPBS and resuspended in cold staining buffer (HBSS containing 2% heat-inactivated FBS) for 10 min blocking on ice. Then, they were stained with the relevant MoAbs for 30 min on ice, washed twice with staining buffer and acquired on the same day on a BD Canto II flow cytometer (Becton Dickinson, Franklin Lakes, N.J.). Data were analyzed using the Pro CellQuest software. γδ T cells were first gated using the forward and side scatter dot plots, and the cell population highly expressing γδ TCR and CD3 was further analyzed for other phenotypic markers or intracellular cytokines.

Synthetic Peptides

HLA-restricted immunodominant peptides derived from NY-ESO-1 and EBV Prombix® pooled peptides were purchased from Proimmune (Oxford, UK). Purities were 279% as indicated by reverse-phase high performance liquid chromatography and mass spectrometry. MACS GMP PepTivator® EBV LMP2A consisted of lyophilized overlapping oligopeptides (mainly 15-mer), covering the sequence of the LMP2A protein of Epstein-Barr virus strain B95-8 [Swiss-Prot Acc. no. P13285] (total purity of 290% as determined by RP-HPLC; Miltenyi).

Tumor Cell Lines

C666-1, Hep3B, DLD-1 and K562 (all except C666-1 were purchased from American Type Culture Collection [ATCC], Manassas, Va.; C666-1 was a gift) were maintained at 37° C., 5% CO₂ in DMEM medium supplemented with 10% defined FBS, 100 units/ml penicillin, 100 units/ml streptomycin and 100 units/ml L-glutamine (all from Life Technologies). C666-1, Hep3B, DLD-1 and K562 tumour lines were derived from nasopharyngeal carcinoma, hepatocellular carcinoma, colorectal carcinoma and myelogenous leukemia, respectively. All tumour cell lines were tested regularly and found to be negative for Mycop/asma infection (Mycoplasma Detection Kit; American Type Culture Collection).

Isolation of Peripheral Blood Mononuclear Cells from Fresh Whole Blood

Peripheral blood mononuclear cells (PBMCs) were prepared from 100 ml of fresh whole blood from healthy volunteers. PBMCs were first separated on Ficoll lymphoprep (Nycomed Pharma, Oslo, Norway; 400× g, 30 min, brake off) and washed twice with HBSS (400× g, 5 min, with brake). Then the PBMCs were resuspended in 90% heat-inactivated defined fetal bovine serum (FBS) and 10% DMSO, and frozen to −80° C. with a controlled-rated freezer. After that, they were transferred to −150° C. liquid nitrogen until ready for use to generate gamma-delta (γδ) T cells, dendritic cells (DCs) or naïve CD4⁺ and CD8⁺ T cells.

Gamma-Delta T Cell Preparation

Cryopreserved PBMCs were rapidly thawed in 37° C. water bath and washed twice with HBSS (400× g, 8 min, with brake) before use. For cell culture media and serum optimization experiments, a total of 1×10⁷ healthy donor PBMCs were seeded into a T25 flasks and cultured for a total of 10 days in either OpTimizer T cell medium (Gibco; supplemented with 1× Optimizer T cell supplement and 100 units/ml HEPES) or Click's medium (Irvine Scientific; supplemented with 100 units/ml HEPES) with different percentages of human AB serum (i.e. 2% or 5%) or 10% heat-inactivated defined fetal bovine serum (FBS). Zoledronic acid (5 μM) was added to the PBMCs on Day 1 and 3 to activate gamma-delta (γδ) T cells, while human recombinant interleukin (IL)-2 (200 IU/ml; clinical grade, Proleukin®) was added to assist in γδ T cell proliferation following zoledronic acid activation. For cytokine optimization experiments, PBMCs were cultured for 10 days in Optimizer T cell media supplemented with 1× Optimizer T cell supplement, 100 units/ml HEPES and 10% heat-inactivated defined FBS. Zoledronic acid (5 μM) was added on Day 1 and 3 together with different combinations of human recombinant cytokines (i.e. IL-2 at 200 IU/ml, IL-7 at 10 ng/ml, IL-15 at 10 ng/ml, IL-18 at 10 ng/ml and IL-21 at 30 ng/ml; all except IL-2 were GMP grade and purchased from CellGenix). At the end of Day 10, unpurified γδ T cells were harvested for evaluation of purity, cell number and phenotypic analysis. In some experiments, γδ T cells were purified with magnetic bead separation following manufacturer's instructions (Miltenyi) and used in tumour cell cytotoxic assays and naïve CD4⁺ and CD8⁺ T cell cocultures.

Monocyte-Derived Dendritic Cell Preparation

Cryopreserved PBMCs were rapidly thawed in 37° C. waterbath, washed twice with HBSS (400× g, 8 min, with brake), resuspended in RPMI medium supplemented with 10% heat-inactivated defined FBS, and seeded at 1×10⁶ cells/ml in 6-well plates (Corning). After 4 hours of incubation at 37° C., 5% CO₂, nonadherent representing lymphocytes were removed by gentle washing and adherent representing monocytes were cultured for a total of 7 days in RPMI medium containing 10% heat-inactivated defined FBS, 500 IU/ml human recombinant granulocyte macrophage colony-stimulating factor (GM-CSF; GMP grade, GellGro) and 250 IU/ml IL-4 (GMP grade, GellGro). On Day 5, fresh GM-CSF and IL-4 were added. On Day 7, these dendritic cells (DCs) were >95% pure (as judged by the absence of CD3- or CD19-expressing lymphocytes and expression of CD1a). The cells had an “immature” phenotype characterized by absence of CD83; low levels of CD86; and moderate levels of HLA-DR, HLA-ABC, and CD40. They showed a typical dendritic cell appearance by light microscopy.

Naïve T Cell Isolation and CFSE-Labeling

Naïve CD4⁺ and CD8⁺ T cells were derived from the nonadherent lymphocyte population after 4 hours of plastic adhesion as described in the DC preparation. The naïve CD4⁺ and CD8⁺ T cells were isolated using magnetic bead separation kit (Miltenyi) following manufacturer's instructions. Then, they were labeled with cell membrane CFSE (carboxyfluorescein diacetate succinimidyl ester) dye (final concentration of 5 μM; Molecular Probes) for 20 min at 37° C. and excess CFSE was adsorbed by adding an equal volume of RPMI medium containing 10% heat-inactivated defined FBS with further 5 min incubation. After that, they were washed once with HBSS (400× g, 8 min, with brake) and used for 2-week cocultures with peptide-pulsed γδ T cells or DCs.

Coculturing CFSE-Labeled Naïve T Cells with Peptide-Pulsed γδ T Cells or DCs

Day 10 purified γδ T cells or Day 7 DCs were pulsed with Epstein-Barr virus (EBV) or NY-ESO-1 Prombix peptides (10 μg/ml; Proimmune) for 2 hours and activated overnight with lipopolysaccharides (LPS) [100 ng/ml; Invivogen]. γδ T cells or DCs were harvested the next day and washed twice with HBSS (400× g, 5 min, with brake) before coculturing with CFSE-labeled naïve CD4⁺ and CD8⁺ T cells (ratio of 10 naïve T cells to 1 γδ T cell or DC). After a week of coculture, viable CD4⁺ and CD8⁺ T cells were restimulated with fresh Day 10 γδ T cells or Day 7 DCs that had been pulsed with relevant peptides and activated with LPS as described above. As controls, unpulsed γδ T cells or DCs were cocultured with CFSE-labeled naïve CD4⁺ and CD8⁺ T cells as described above. At the end of 2 weeks, CD4⁺ and CD8⁺ T cells were assessed for their proliferation as visualized by the dilution of CSFE staining on flow cytometry. These CD4⁺ and CD8⁺ T cells were also evaluated for their phenotypes and antigen-specificities with flow cytometry and pentamer staining, respectively.

Large-Scale Coculturing of Peripheral Blood Lymphocytes Cells with EVB-LMP2 Peptide-Pulsed γδ T Cells or DCs

To mimic the large-scale procedure we would perform in the clinic, we obtained 300 ml of whole blood from healthy volunteers to isolate sufficient PBMCs, PBLs and monocytes for generating γδ T cells, responder T cells and DC, respectively. One-third of the PBMCs were used for generating γδ T cells, while the rest were plated to obtain monocytes and PBLs. The γδ T cells and DCs were generated as described earlier in the methods and materials. For the coculture, Day 10 purified γδ T cells or Day 7 DCs were pulsed with MACS® GMP PepTivator® EBV LMP2A (a pool of mainly ˜15mer overlapping oligopeptides covering the sequence of EBV LMP2A protein; final concentration of 0.6 nmol or ˜1 μg of each peptide per ml; Miltenyi) for 2 hours and activated overnight with lipopolysaccharides (LPS) [100 ng/ml; Invivogen] for γδ T cells, or with proinflammatory cytokines (prostaglandin 2A, TNF-α, IL-1β and IL-6; all from Cellgro) for DCs overnight. The next day, γδ T cells or DCs were harvested, washed twice with HBSS (400× g, 5 min, with brake) before coculturing with PBLs at a ratio of 10 naïve PBLs to 1 γδ T cell or DC). After a week of coculture, viable PBLs were restimulated with fresh Day 10 γδ T cells or Day 7 DCs that had been pulsed with relevant peptides and activated with LPS or proinflammatory cytokines as described above. During the coculture, IL-7 and IL-15 were added at 10 ng/ml each on Day 2 and every 3 days thereafter to support T cell growth. At the end of 2 weeks, PBLs were assessed for exhaustion and activation markers, and IFN-γ secretion in response to PepTivator® EBV LMP2A peptide pool.

Phenotypic Analysis

For phenotype studies, cells were resuspended in cold staining buffer (HBSS containing 2% heat-inactivated defined FBS) for 10 min blocking at 4° C. Then, the cells were incubated with the relevant MoAbs for 30 min at 4° C. Following that, the cells were washed twice (500× g, 5 min, with brake) with staining buffer and analyzed immediately with BD Canto II flow cytometer (Becton Dickinson). Data were analyzed using Data were analyzed using the Pro CellQuest software. For γδ T cell analysis, the relevant cells were first gated using the forward and side scatter dot plots and the cell population that highly expressing both γδ T cell receptor (TCR) and CD3 was further analyzed for HLA-ABC, HLA-DR, CD40, CD80, CD83, CD86, ICAM-1, CCR5, CCR6, CCR7, NKG2D, PD-1, CTLA-4, TIM-3, LAG-3 toll-like receptor (TLR)-1, TLR-2, TLR-3, TLR-4, TLR-5, TLR-7 and TLR-9. For CD4⁺ and CD8⁺ T cell analysis, the relevant cell population that highly expressing al TCR, CD3 and CD4/CD8 was further analyzed for effector, effector memory, central memory, exhaustion (PD-1, CTLA-4, TIM-3, LAG-3) and FOXP3 regulatory T cell markers. For DC analysis, relevant cell population that highly expressing CD11c and HLA-DR was further analyzed for CD40, CD80, CD83, CD86 and ICAM-1. For tumour cell line analysis, relevant cell population was gated with forward and side scatter dot plots and further analyzed for MICA, MICB and BTN3A1 expressions.

Intracellular Cytokine Staining

γδ T cells were stimulated with phorbol myristate acetate (PMA) [50 μg/ml] and ionomycin (100 μg/ml) [both from Sigma-Aldrich] to evaluate their cytokine profile. After the 1^{st} hour of the total 5 hour incubation, γδ T cells were pelleted by centrifugation (500×g, 5 min with brake) and GolgiStop containing brefeldin A (1000× dilution according to manufacturer's instructions; BD Pharmingen) was added to the cells for the remainder of the incubation period. After that, the cells were harvested and stained for FITC-conjugated anti-γδ TCR and Pacific Blue-conjugated anti-CD3 for 30 min at 4° C. This is followed by fix-permeabilization treatment (BD) for 30 min at 4° C. to stain intracellularly for IFN-γ, TNF-α, and IL-17. Then, the cells were washed twice with staining buffer (HBSS containing 2% heat-inactivated FBS) and interrogated on the same day with BD Canto II flow cytometer (Becton Dickinson). The data was analyzed with Pro CellQuest software. For IL-10 intracellular staining, GolgiStop containing brefeldin A was added in the 1^{st} hour of the total 12 hour incubation, stained and analyzed as described above. γδ T cells that were positive for intracellular IFN-γ, TNF-α, IL-10 or IL-17 were expressed as a percentage of the gated γδ TCR⁺ CD3⁺ T cells. γδ T cells not stimulated with PMA and ionomycin were evaluated the same way to account for background cytokine secretions.

Pentamer Staining

CD4⁺ and CD8⁺ T cells that had been stimulated with peptide-pulsed γδ T cells or DCs for 2 weeks were evaluated for their antigen-specificities with pentamer staining. 1×10⁶ T cells per group were washed once with staining buffer (HBSS with 2% FCS) and stained with a phycoerythrin (PE)-conjugated HLA-A*1101-restricted EBV LMP2 pentamer (abbreviated p-EBV LMP2₃₆₉₋₃₇₇; ProImmune) or HLA-A*2401-restricted NY-ESO-1 pentamer (abbreviated p-NY-ESO-1₆₉₋₃₇₇; ProImmune) for 20 min at 370C. T cells were then counterstained with anti-CD8-APC or anti-CD4-APC-Cy7 for 30 min at 4° C. Following that, the cells were washed twice with staining buffer and analyzed by flow cytometry, gating on CD4⁺ or CD8⁺ cells. T cells that were double positive for CD4/CD8 and pentamer were expressed as a percentage of the total number of CD4⁺/CD8⁺ T cells gated.

Tumor Cytotoxic Assay

DELFIA® EuTDA Cytotoxicity assay was used to evaluate tumour cell lysis by γδ T cells. Briefly, γδ T cells were seeded in 96-well V bottom plates in graded numbers (i.e. 1×10⁵, 5×10⁴, 2.5×10⁴ per well). Then, tumor cells (i.e. C666-1, Hep3B, DLD-1 and K562) were added to the γδ T cells at 5×10³ cells per well. The cells were cocultured for a total of 2 hours at 37° C., 5% CO² before the supernatants were analyzed for lysis of labeled tumor cell targets according to the manufacturer's protocol. All assays were performed in triplicate. The measured fluorescence signal was correlated directly with the amount of lysed cells and the results were expressed as % tumor cell lysis by γδ T cells.

Cytokine and Chemokine Array Analysis

γδ T cells were cocultured with different tumor lines (i.e. C666-1, Hep3B, DLD-1 and K562) at a ratio of 20 effector γδ T cells (1×10⁵) to 1 tumor cells (5×10³) in 96-well V bottom plate for 24 hours at 37° C., 5% CO₂. Then, the coculture supernatants were collected and evaluated for granzymes A and B, perforin, granulysin, IFN-γ, IL-17, IL-8, Eotaxin, IP-10, MIG, GRO A, MIP-3A, I-TAC, MCP-1, RANTES, MIP-1A, MIP-1B and ENA-78 with Biolegend Legendplex® cytometric bead array (Biolegend) and BD Canto II flow cytometer according to the manufacturer's protocol. As negative controls, supernatants from tumor cells or γδ T cells alone were evaluated. All assays were performed in duplicate. The data was presented as μg/ml or ng/ml.

Statistical Analysis

Means for different experimental groups were analyzed from 3 to 6 independent experiments (i.e. DCs from 3 to 6 different individuals). The analysis of significance was carried out using unpaired Student's t-tests or one-way ANOVA. A significance level of 0.05 or less was considered statistically significant. Analyses were conducted in GraphPad Prism.

### Example 2: Results and Discussion

Optimizer T Cell Medium is Superior than Click's Medium in Generating a Higher Yield and Purity of Peripheral Blood Derived-γ9δ2 T Cells.

Click's and Optimizer T cell media are two widely used clinical grade serum-free defined media for expanding large numbers of tumor-infiltrating T cells (TILs) or activated tumor-specific CD4⁺ and CD8⁺ T cells. However, no clinical trials and preclinical studies had explored the use of these media in generating γδ T cells from peripheral blood. Serum derived from bovine or human provides a good source of nutrients for rapidly expanding CD4⁺ and CD8⁺ T cells. Autologous serum from cancer patients is not an ideal source as it could contain high levels of inhibitory cytokines such as IL-10, IL-6 or transforming growth factor (TGF)-β to suppress the proliferation and function of γδ T cells. A suitable alternative is the pooled normal human AB serum which is free from T cell inhibitory cytokines and infectious agents. Defined FBS is also available for clinical use. As opposed to normal FBS, defined FBS is certified free from bovine-related infectious agents and other contaminants that could adversely affect T cell generation. We have successfully used defined FBS to generate large numbers of cytotoxic T lymphocytes (CTLs) specific to Epstein-Barr virus (EBV) in a Phase I/II trial of nasopharyngeal carcinoma (NPC) [21].

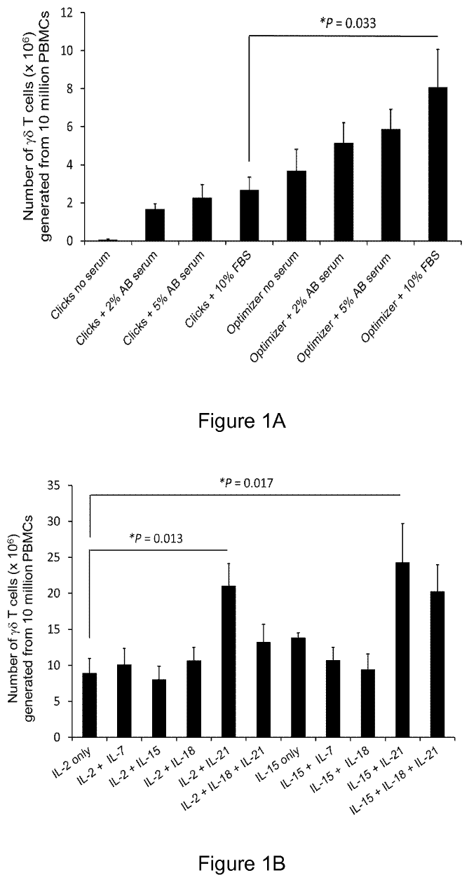

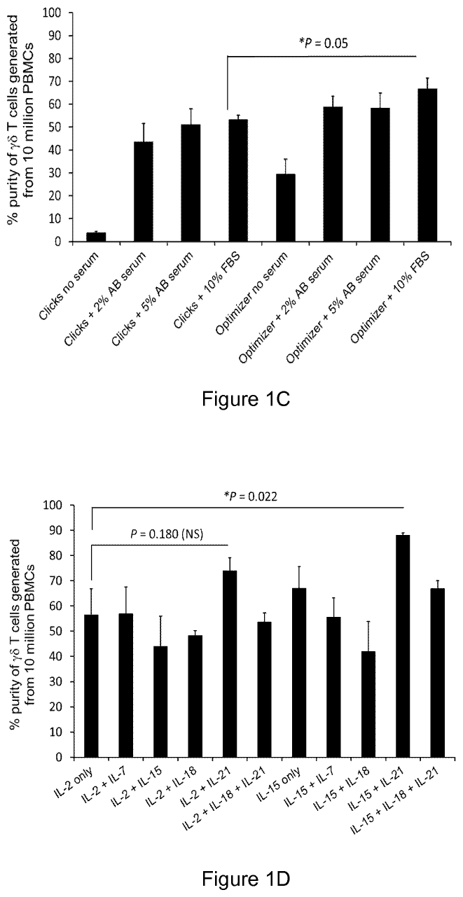

In this study, we evaluated different media and serum combinations for culturing peripheral blood-derived γ9δ2 T cells. We compared Click's and Optimizer T cell medium supplemented with 2% or 5% pooled human AB serum, or 10% defined FBS. Using 10 million cryopreserved PBMCs as the starting population, we determined the yield and percentage purity of the generated γδ T cells after 10 days of culture. In summary, we found that Optimizer T cell medium was superior than Click's medium in supporting γδ T cell expansion whether serum-free or serum-supplemented (FIG. 1A; 3-8×10⁶ γδ T from Optimizer T cell medium compared to 0.5-2.5×10⁶ γδ T from Click's medium). The addition of 2% or 5% pooled human AB serum to either Click's or Optimizer T cell medium enhanced the proliferation of the γδ T cells. However, the addition of 10% defined FBS to Optimizer T cell medium generated the highest number of γδ T cells compared to Click's medium supplemented with 10% defined FBS (FIG. 1A; 8×10⁶ γδ T cells compared to 2.5×10° γδ T cells from Click's medium, P=0.033, Student paired t-test, *highly significant). We observed that purity of ex vivo generated γδ T cells was improved with the addition of pooled human AB serum or defined FBS to the culture medium (FIG. 1C). The highest purity of γδ T cells was obtained with Optimizer T cell medium supplemented with 10% defined FBS (FIG. 1C; 65% compared to 50% from Click's medium plus 10% defined FBS, P=0.033, Student paired t-test, *highly significant). Based on these results, we selected the novel combination of Optimizer T cell media supplemented with 10% defined FBS as the optimal combination for further evaluation.

Recombinant Human IL-21 Further Enhanced the Purity and Yield of Ex Vivo Generated γ9δ2 T Cells.

IL-2 and IL-15 are widely used cytokines for ex vivo expansion of CD4⁺ and CD8⁺ T cells. IL-2 is commonly used for generating γδ T cells in the clinics (22, 23), while IL-15 is known for inducing the proliferation of memory CD4⁺ and CD8⁺ T cells (24). IL-7 is required for the homeostatic maintenance and proliferation of naïve CD4⁺ and CD8⁺ T cell (22, 23). IL-18 has been shown to elicit a stronger IFN-γ response from γδ T cells (25), while IL-21 could enhance the cytotoxic activity of ex vivo generated γ9δ2 T cells (26). All these cytokines are available in GMP grade for clinical use. The synergistic effects of these cytokines on the generation of Vγ9Vδ2 T cells have not been explored in detail. Thus, we analyzed the effect of these recombinant human cytokines on the yield and purity of the generated γ9δ2 T cells from a starting population of 10 million cryopreserved PBMCs. We observed that IL-15 alone or in combination of IL-7, IL-18 or IL-21 was more superior in expanding γδ T cells than IL-2 alone or in combinations with the above mentioned cytokines (FIG. 1B). We also observed that recombinant human IL-21 significantly increased the number of expanded γδ T cells when used in combination with IL-2 or IL-15 (FIG. 1B; P=0.017 and 0.013, respectively, when compared to IL-2 alone, *highly significant). The purity of the generated γδ T cells was also significantly improved when cultured in the presence of IL-15+IL-21 (88.1%) compared to IL-2 alone (56.5%) [FIG. 1D; P=0.022, Student paired t-test, *highly significant]. Although IL-2+IL-21 generated a higher % purity of γδ T cells (74%), it did not reach significance when compared to culturing with IL-2 alone (FIG. 1D; P=0.180, Student paired t-test, *NS=not significant). On the other hand, culturing γ9δ2 T cells with IL-21 alone resulted in low expansion of the cells (15%) suggesting that IL-21 was not an essential growth factor for γ9δ2 T cells ex vivo (data not shown). Therefore, the novel combination of recombinant human IL-21 with IL-15 or IL-2 was beneficial for improving the yield and purity of ex vivo generated γ9δ2 T cells.

Ex Vivo Generated γ9δ2 T Cells Exhibited Desirable Antigen Presentation and Effector Phenotypic Markers and were Highly Proinflammatory Upon Activation.

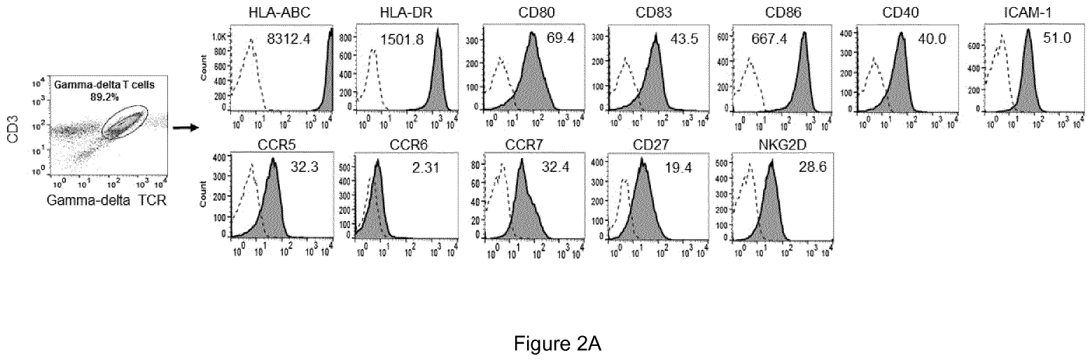

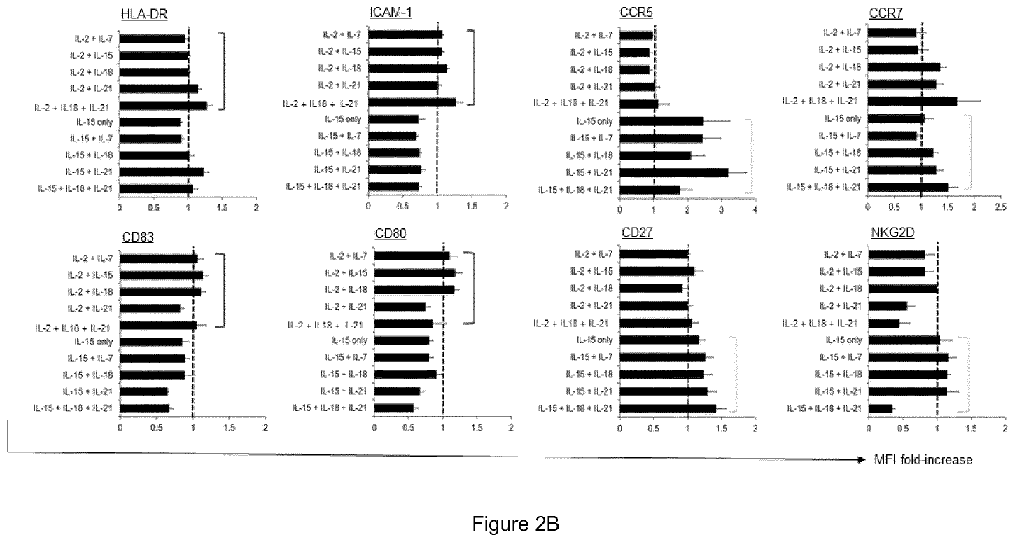

Next, we investigated the effect of different cytokine combinations in influencing the phenotypes and cytokine profiles of the generated γ9δ2 T cells. In FIG. 2A, we showed that the γδ T cells generated in the presence of IL-15 and IL-21 highly expressed antigen presentation markers (i.e. HLA-ABC and HLA-DR), T cell costimulation markers (i.e. CD80, CD83, CD86, CD40 and ICAM-1), as well as effector markers (i.e. CCR5, CCR6, CCR7, CD27 and NKG2D). This phenotypic profile was representative of all the γδ T cells produced under the different cytokine combinations tested (i.e. IL-2 or IL-15 alone or in combinations with IL-7, IL-18 or IL-21; see FIG. 2B), indicating that they had the potential to perform multiple functions of antigen presentation, T cell costimulation and direct tumor cell cytolysis. No study had performed a detailed phenotypic analysis of the above mentioned markers on ex vivo generated γδ T cells. Thus, we were the first to describe the simultaneous expression of these markers on the γδ T cells generated from different novel culture conditions. This is an important finding as it allows us the opportunity to manipulate these γδ T cells ex vivo to maximize their anti-tumor properties. Interestingly, we observed that the γδ T cells produced in the presence of IL-2 alone or in combination with other cytokines expressed a higher level of antigen presentation markers compared to γδ T cells produced in the presence of IL-15 alone or in combination with other cytokines (FIG. 2B; mean fluorescence intensity (MFI) of HLA-DR, ICAM-1, CD83 and CD80 were shown as fold-increase normalized against IL-2 alone condition, indicated in the brackets). On the other hand, γδ T cells generated in the presence of IL-15 showed higher effector CCR5, CCR7, CD27 and NKG2D makers compared to γδ T cells generated in the presence of IL-2 (FIG. 2B; fold-increase MFIs normalized against IL-2 alone condition, indicated in the brackets). This finding suggested that we could potentially skew the ex vivo generated γδ T cells to exhibit a stronger antigen presentation or effector tumor cytolysis function through the selective use of IL-2 and IL-15.

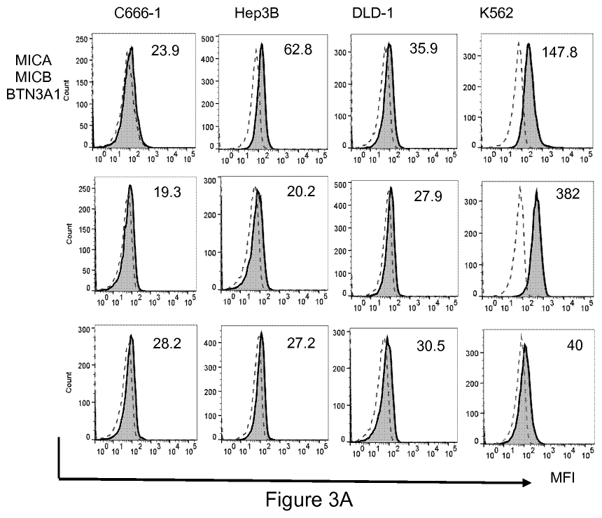

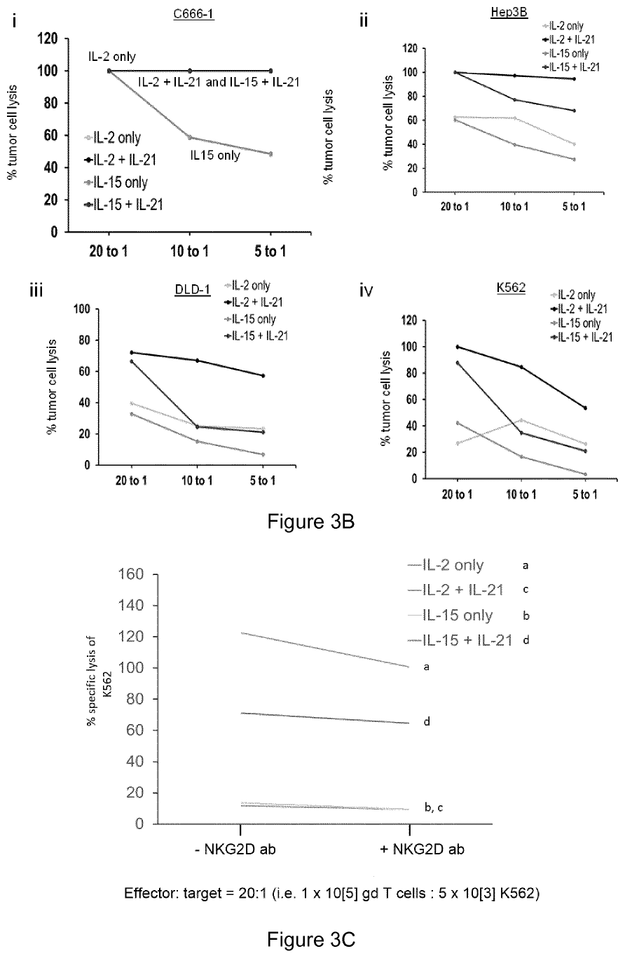

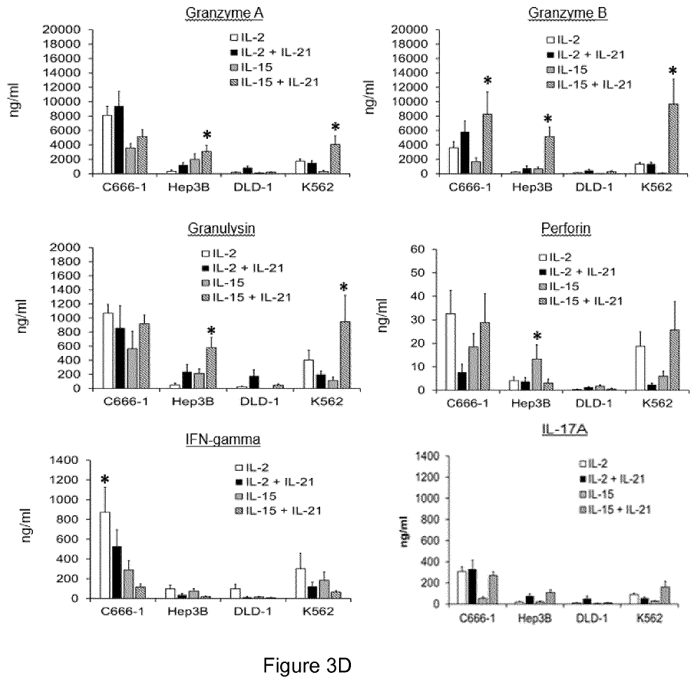

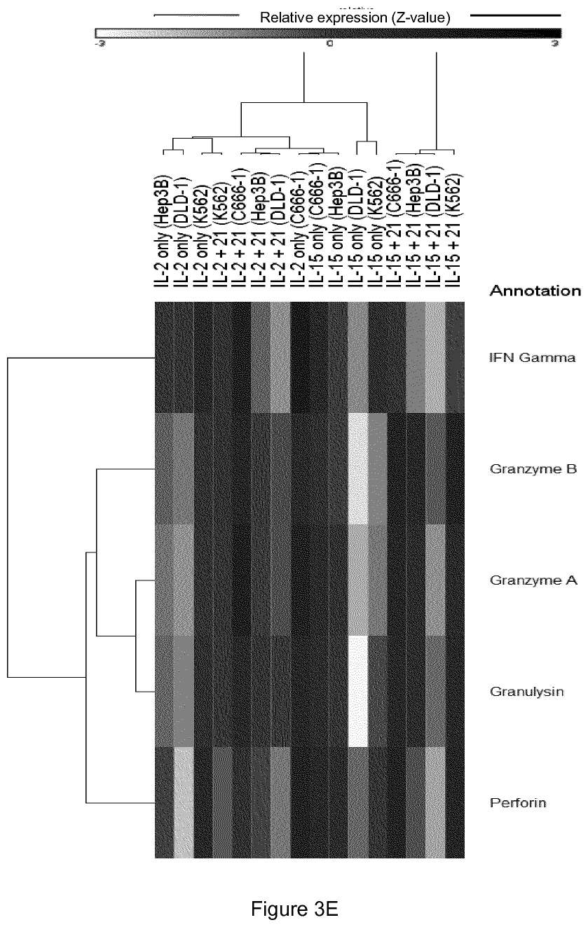

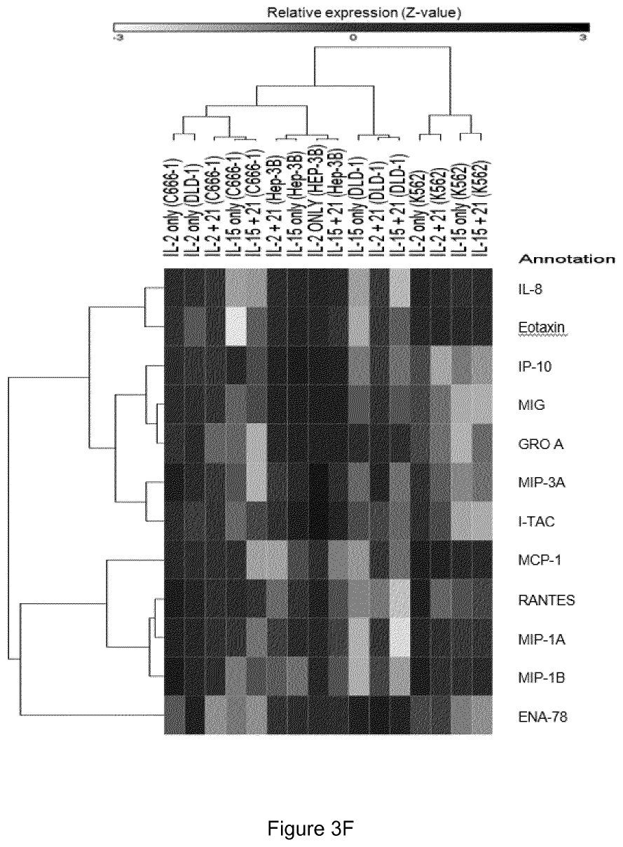

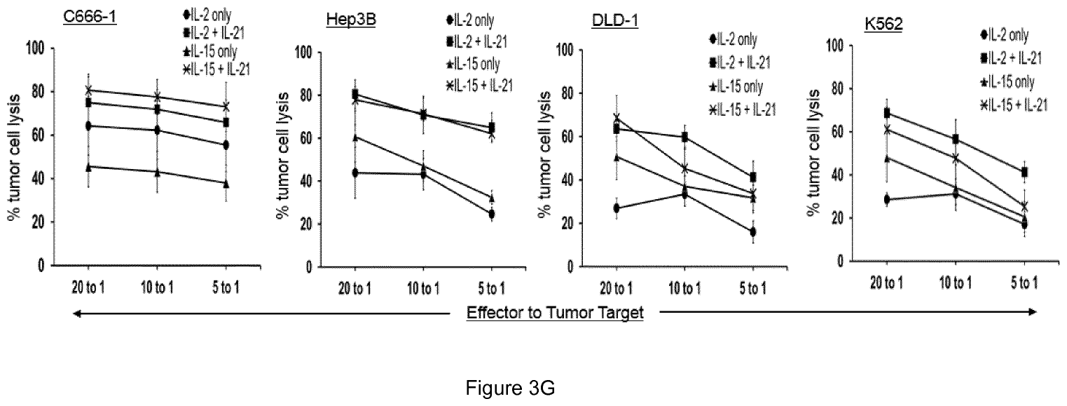

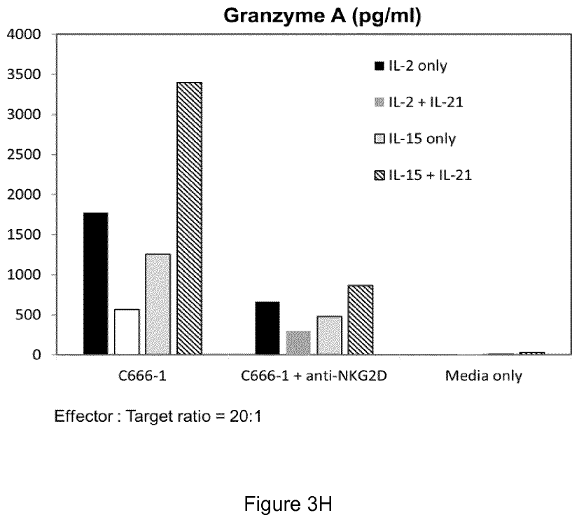

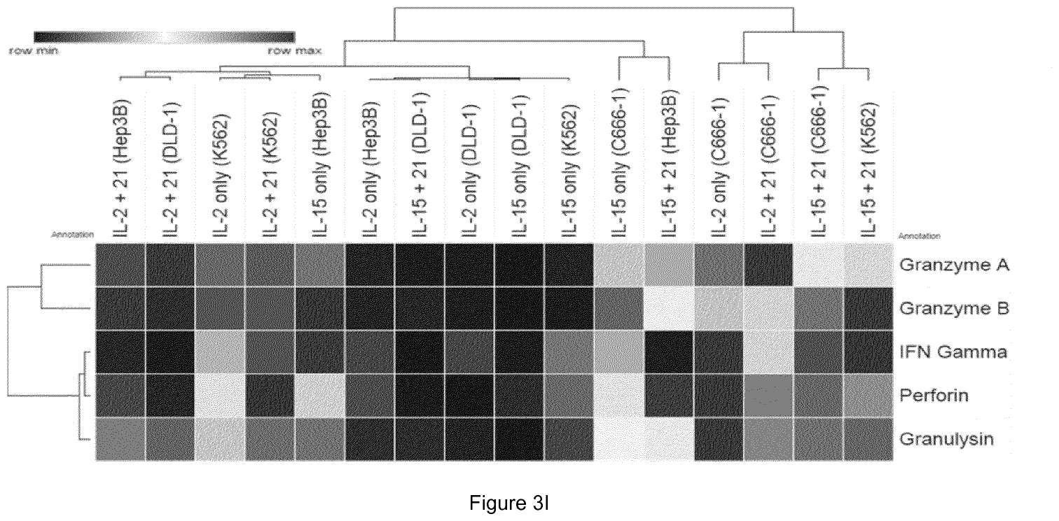

We further evaluated the cytokine profile of these ex vivo generated γδ T cells by activating them with PMA and ionomycin. After 5 hours of stimulation, we performed intracellular cytokine staining to determine the % of γδ T cells expressing IFN-γ, TNF-α, IL-17 and IL-10. We found that a high % of γδ T cells could be activated by PMA and ionomycin to produce proinflammatory IFN-γ only (54.82±12.09% to 66.42±4.68%) or TNF-α only (54.1±11.6% to 66.9±8.3%) regardless of their cytokine culture condition (Table 1; columns 1 and 2). A smaller % of γδ T cells were also capable of producing both IFN-γ and TNF-α upon activation (Table 1; 17.7±5.6% to 48.6±9.4%, column 3). Notably, very small % of these ex vivo generated γδ T cells produced IL-17 (0±0% to 1.08±0.72%) and IL-10 (0±0.02 to 0.51±0.28%), suggesting that they preferentially elicit proinflammatory T helper (Th)-1 and cytotoxic T cell (CTL) responses. This finding was important as both IL-17 and IL-10 have been implicated in assisting tumor progression, thus low expression of these cytokines from γδ T cells was preferred for generating a strong anti-tumor response. The following groups were selected for further functional analysis—i.e. IL-2 alone, IL-2+IL-21, IL-15 and IL-15+IL-21. We selected the IL-2 alone group because all the published clinical trials so far had used IL-2 alone for γδ T cell expansion. This group served as the baseline response for comparison of all the functional analysis in our study. The other three groups (IL-15 alone, IL-2+IL-15 and IL-15+IL-21) were selected because they consistently gave one of the highest yield and % purity of γδ T cells compared to IL-2 alone and other groups (see FIG. 1). In addition, the γδ T cells generated from these groups showed desirable expressions of both antigen presentation and effector makers (FIG. 2A), as well as favorable proinflammatory cytokine profiles of high IFN-γ and TNF-α (FIG. 3A). As we observed a difference in the antigen presentation and effector marker expressions between IL-2 and IL-15 generated γδ T cells (FIG. 2B), these four groups also allow us to compare their antigen presentation and effector functions.

TABLE 1
Percentage of gamma-delta T cells producing IFN-γ, TNF-α, IL-17
and IL-10 following PMA and Ionomycin stimulation

| | % gamma-delta T cells producing cytokines following PMA and ionomycin stimulation | | | | |
|---|---|---|---|---|---|
| Cytokine condition | IFN-γ | TNF-α | IFN-γ + TNF-α | IL-17A | IL-10 |
| IL-2 only | 54.82 ± 12.09 | 63.39 ± 11.4 | 17.7 ± 5.6 | 0 ± 0 | 0.03 ± 0.04 |
| IL-2 + IL-7 | 56.91 ± 12.13 | 63.88 ± 10.3 | 14.8 ± 3.3 | 0 ± 0 | 0.05 ± 0.07 |
| IL-2 + IL-15 | 65.62 ± 6.47 | 64.04 ± 9.7 | 32 ± 8.9 | 0 ± 0 | 0.02 ± 0.06 |
| IL-2 + IL-18 | 62.79 ± 12.57 | 66.90 ± 8.3 | 48.6 ± 9.4 | 4.11 ± 2.4 | 0.12 ± 0.1 |
| IL-2 + IL-21 | 66.01 ± 5.09 | 64.07 ± 11.9 | 21.8 ± 7.3 | 0.74 ± 0.17 | 0.33 ± 0.15 |
| IL-2 + IL-18 + IL-21 | 62.89 ± 7.70 | 62.58 ± 10.7 | 26 ± 5.8 | 0 ± 0 | 0.06 ± 0.07 |
| IL-15 only | 66.42 ± 4.68 | 61.70 ± 8.1 | 41.7 ± 4.6 | 0 ± 0 | 0.02 ± 0.01 |
| IL-15 + IL-7 | 55.78 ± 14.30 | 61.90 ± 7.4 | 41.4 ± 6.3 | 0 ± 0 | 0 ± 0.02 |
| IL-15 + IL-18 | 55.46 ± 5.76 | 57.23 ± 10.1 | 18.5 ± 4.8 | 0.61 ± 1.40 | 0.02 ± 0.01 |
| IL-15 + IL-21 | 61.53 ± 6.72 | 56.69 ± 6.5 | 35.9 ± 3.1 | 1.08 ± 0.72 | 0.51 ± 0.28 |
| IL-15 + IL-18 + IL-21 | 62.70 ± 4.66 | 54.10 ± 11.6 | 25.1 ± 5.4 | 0 ± 0 | 0.06 ± 0.01 |

Ex Vivo Generated γ9δ2 T Cells were Highly Efficient in Killing Broad Range of Tumor Cells Via NKG2D Ligand Recognition and Displayed Differential Cytokine and Chemokine Profiles.

To determine if the ex vivo generated γ9δ2 T cells could directly recognize and lyse tumor cells, we cocultured the purified γδ T cells with different tumor types at varying effector to tumor target ratios (i.e. 20 to 1, 10 to 1 and 5 to 1) as described in the Materials and Methods section. We chose four cell lines derived from different tumor types—C666-1 (nasopharyngeal carcinoma), Hep3B (hepatocellular carcinoma), DLD-1 (colorectal carcinoma) and K562 (myeloid leukemia) for analysis—and determined their MICA, MICB and BTN3A1 expressions which were ligands for direct tumor cytolysis by γδ T cells via their NKG2D and γδ T cell receptors (TCRs). We determined that all the tumor lines expressed MICA, MICB and BTN3A1, with K562 line expressing the highest levels of these three ligands amongst the four tumor cell lines (FIG. 3A; dotted open and solid shaded histograms represented isotype control and tests, respectively). The corrected MFIs were indicated in the upper right hand corner.

In FIG. 3B, we evaluated the % lysis of tumor cells by γδ T cell in a 2 hour assay, and FIG. 3G shows further analysis. Strong tumor cytolysis by γδ T cells was observed in 2 hours, indicating that these ex vivo generated γδ T cells were highly capable of recognizing and killing a broad range of tumor types. Stronger tumour cytotoxic activities were observed from γδ T cells that were generated in combination with IL-21 than those generated with IL-2 or IL-15 only (FIGS. 3C; and 3G). These IL-21 generated γδ T cells were also more cytolytic towards virus-expressing C666-1 and Hep3B lines than non-virus expressing DLD-1 and K562 lines (FIG. 3B). The results suggested that IL-21 endowed a stronger cytolytic capability to the γδ T cells. We determined that this direct tumor cell killing by the generated γδ T cells was through NKG2D-ligand (MICA/MICB) recognition as reduced K562 cytolysis (data not shown) and reduced production of granzyme A (FIG. 3H) was seen when the γδ T cells were blocked with NKG2D blocking antibody.

We further evaluated the mode of direct tumor cytolysis of γδ T cells by measuring their secreted granzymes A and B, perforin, granulysin and IFN-γ following 24 hours incubation with the above tumor cell lines. All the γδ T cells were able to produce granzymes A and B, granulysin, perforin and IFN-γ (FIGS. 3D and 3I). Similar to CD8⁺ CTLs, these ex vivo generated γδ T cells used granzymes A and B, perforin and granulysin to target and lyse tumor cells. Cluster analysis of relative expressions (Z-values) revealed that γδ T cells generated in the presence of IL-2 produced a higher level of IFN-γ in response to live tumour cells than γδ T cells that were generated in the presence of IL-15 (FIGS. 3D and 3I). It also showed that γδ T generated with IL-2+IL-21 and IL-15+IL-21 preferentially use granzyme A and B, respectively, for tumour lysis. The presence of IL-21 seemed to enhance the production of granzymes and granulysin from the γδ T cells as lower levels were observed from IL-2 only and IL-15 only groups. This observation was in-line with the higher tumour cytolysis observed with IL-2+IL-21 and IL-15+IL-21 groups as shown in FIGS. 3B, 3C and 3G. The colorectal carcinoma line, DLD-1, induced an overall reduced production of granzymes, granulysin, perforin and IFN-γ in the γδ T cells regardless of the latter culture conditions. This could be due to the intrinsic factors of DLD-1 line or colorectal carcinoma lines. The levels of granzymes, granulysin and perforin were quantified and are shown in FIG. 3D.

Next, we used cluster analysis to evaluate the chemokine profiles of γδ T cells in response to live tumor cells (FIG. 3F). Interestingly, their chemokine expressions were strongly influenced by the type of tumor cells that they were exposed to. When exposed to K562 and Hep3B tumor cells, a strong Th2 chemokine profile of high IL-8 and eotaxin was observed in all the γδ T cells regardless of their cytokine culture conditions. IL-8 and eotaxin were involved in stimulating humoral responses. In addition, IL-8 was implicated in angiogenesis, metastasis and recruitment of tumor-associated macrophages (TAMs) [27]. Amongst the 4 tumor lines, Hep3B stimulated the strongest amount of GRO-α which was shown to promote angiogenesis and metastasis [28], as well as a chemoattractant for neutrophils [29]. Hep3B also stimulated the most MIP-3a from γδ T cells, especially those of IL-2 groups. MIP-3a was a known chemoattractant for pro-tumorigenic Th17 cells and TAMs [27, 30]. K562 stimulated the least production of MIP-3a from γδ T cells regardless of their cytokine culture conditions. In addition, K562 stimulated the strongest MCP-1 (CCL 2) production from γδ T cells amongst the 4 tumor lines. MCP-1 helped to activate NK cells and recruit CTLs into the tumours [31-32]. On the other hand, it could promote cancer metastasis by recruiting myeloid-derived suppressor cells (MDSCs) and T regulatory cells (Tregs) via the nitration of CCL2 by reactive nitrogen species in the tumour microenvironment [33]. Improved CTL therapy had been observed through blocking the nitration of CCL2 [34]. K562 and Hep3B also stimulated the production of Th1 chemokines from the γδ T cells. Interestingly, K562 preferentially induced Th1-associated MIP-1α and MIP-1β that helped to recruit NK cells and pre-cursor DCs into the tumor, and naïve CD8⁺ T cells to the antigen-dependent clusters of DCs and CD4⁺ T cells for memory CD8⁺ T effector cell differentiation [35]. MIP-1α is also utilized by APCs like DCs to recruit CD8⁺ CTLs [36]. We observed that γδ T cells from IL-2 groups produced more MIP-1α and MIP-1β than those from IL-15 groups. On the other hand, Hep3B preferentially induced Th1-related IP-10, MIG and I-TAC that were indispensable for extravasation of mature cytotoxic effectors and TILs into the tumors for successful adoptive T cell therapy as well as being angiostatic [37-38]. When exposed to C666-1 and DLD-1, the γδ T cells downregulated their IL-8 and eotaxin productions especially in the IL-15 groups. We observed that C666-1 stimulated productions of MCP-1, RANTES, MIP-1α and MIP-1β for its Th1 responses as opposed to Hep3B that preferentially stimulated IP-10, MIG and I-TAC. Nevertheless, C666-1 line was capable of inducing IP-10, MIG and I-TAC from γδ T cells. We observed that MCP-1 and RANTES productions were downregulated in γδ T cells that were cultured in the presence of IL-21 compared to those that were cultured only with IL-2 or IL-15. As shown above, ex vivo generated γδ T cells displayed differential chemokine profiles towards different tumor types. Thus, we could efficiently use γδ T cell-based immunotherapy to target tumor types that induced a stronger Th1 chemokine responses. Conversely, we could augment γδ T cell therapy with immunomodulating therapies to revert their Th2 chemokine response towards certain tumor types to Th1-priming responses.

Ex Vivo Generated γ9δ2 T Cells were More Efficient than Monocyte-Derived Dendritic Cells in Stimulating the Proliferation of Naïve CD4⁺ and CD8⁺ T Cells.

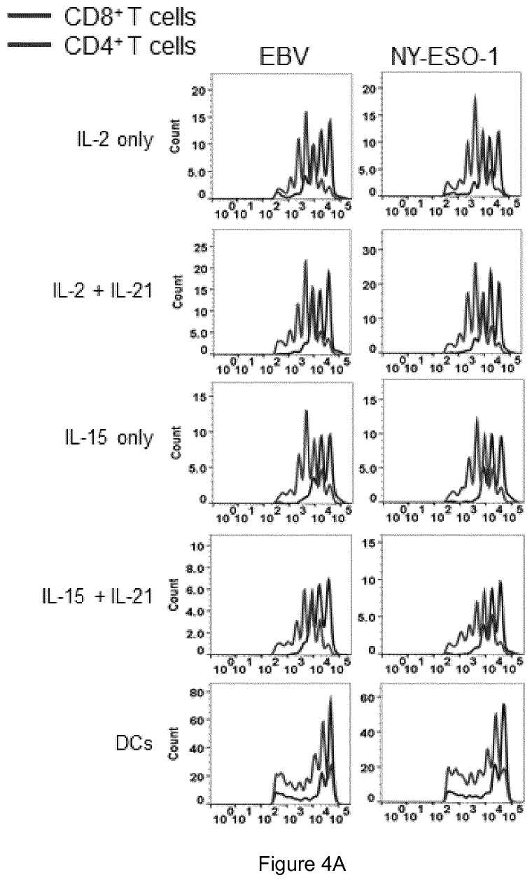

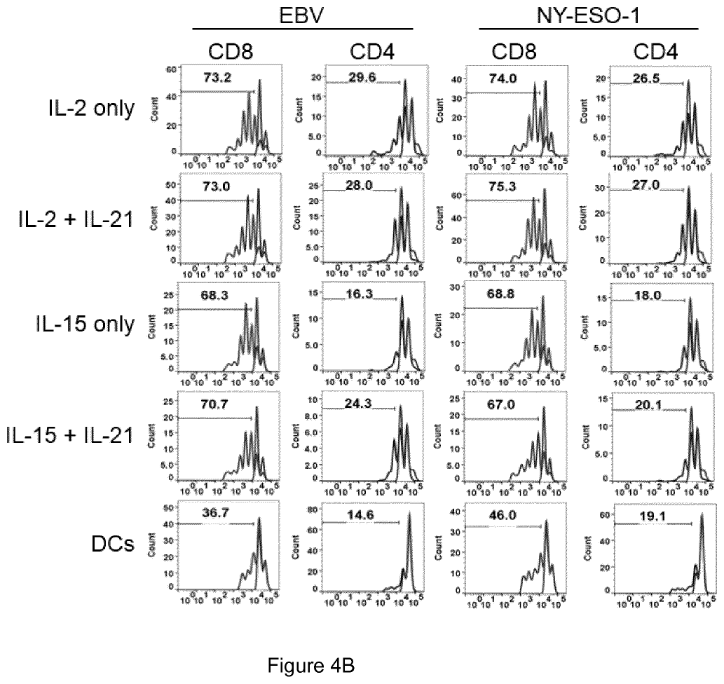

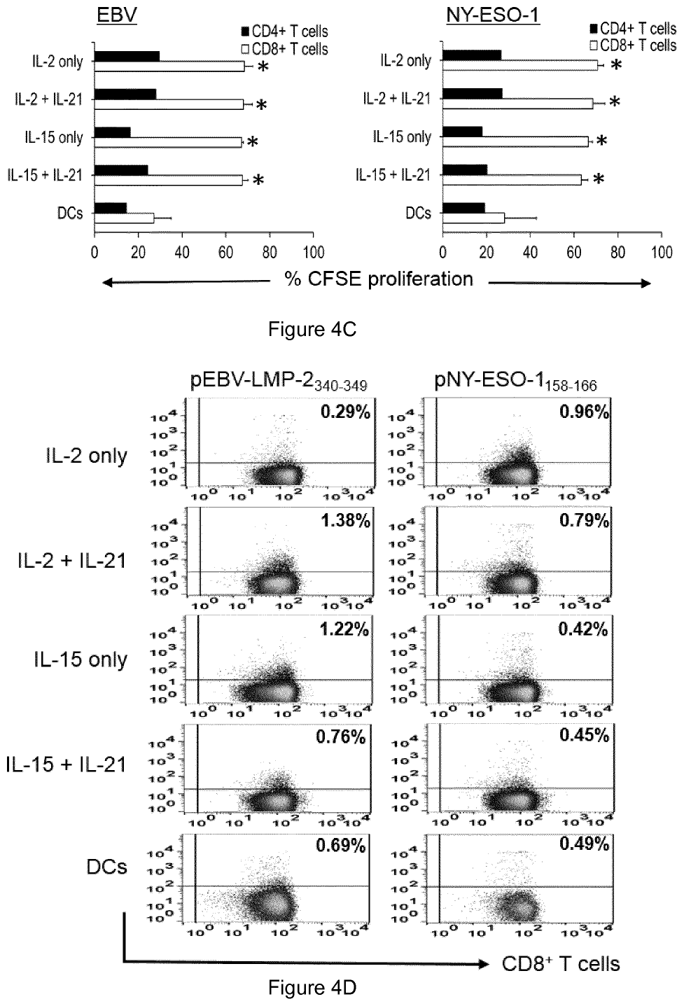

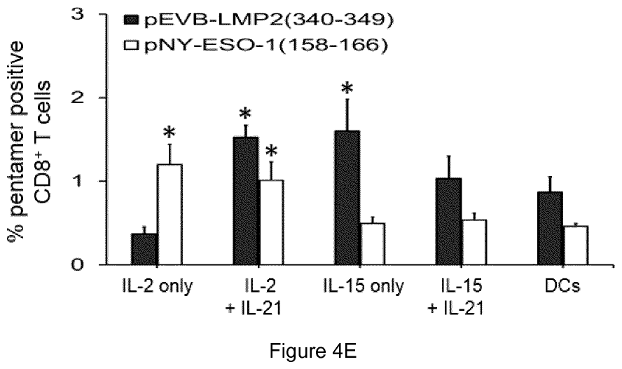

We investigated whether ex vivo generated γ9δ2 T cells could act as antigen-presenting cells to stimulate the proliferation of naïve CD4⁺ and CD8⁺ T cells in cocultures. To test this, we pulsed the γδ T cells with MHC Class I-restricted peptides derived from either EBV or NY-ESO-1 (a tumor-associated antigen) and cocultured with CFSE-labeled naïve CD4⁺ and CD8⁺ T cells for two weeks. At the end of two weeks, we observed that that peptide-pulsed γδ T cells, regardless EBV or NY-ESO-1 derived, stimulated a more robust proliferation of naïve CD4⁺ and CD8⁺ T cells than Day 7 ‘classical’ monocyte-derived dendritic cells pulsed with the same peptides (FIGS. 4A and 4B; Each peak represented a round of T cell proliferation). The percentage of cells determined to be proliferating is shown in FIG. 4B. Significantly higher % of naïve CD8+ T cells proliferated in the presence of peptide-pulsed γ9δ2 T cells (>60%) compared to peptide-pulsed DCs (<30%). Similar results were also observed for naïve CD4+ T cells (FIG. 4C). We further observed that significantly more EBV- and NY-ESO-1-specific CD8+ T cells were detected following simulation of the naïve T cells with peptide-pulsed γδ T cells compared to peptide-pulsed monocyte-derived DCs (FIG. 4D). We found that γδ T cells generated with IL-2+IL-21 or IL-15 only stimulated the highest number of pentamer EBV-LMP2340-349 specific CD8+ T cells, while γδ T cells generated with IL-2 only stimulated the highest number of pentamer NY-ESO-11158-166 specific CD8+ T cells (FIG. 4E).

Lower % of CD4⁺ and CD8⁺ T Cells Expressed PD-1, CTLA-4, TIM3 and LAG3 Exhaustion Phenotypes Following Stimulation with 7962 T Cells Pulsed with EBV-LMP2A Pooled Peptides Compared to DCs Pulsed with the Same Peptide Pool.

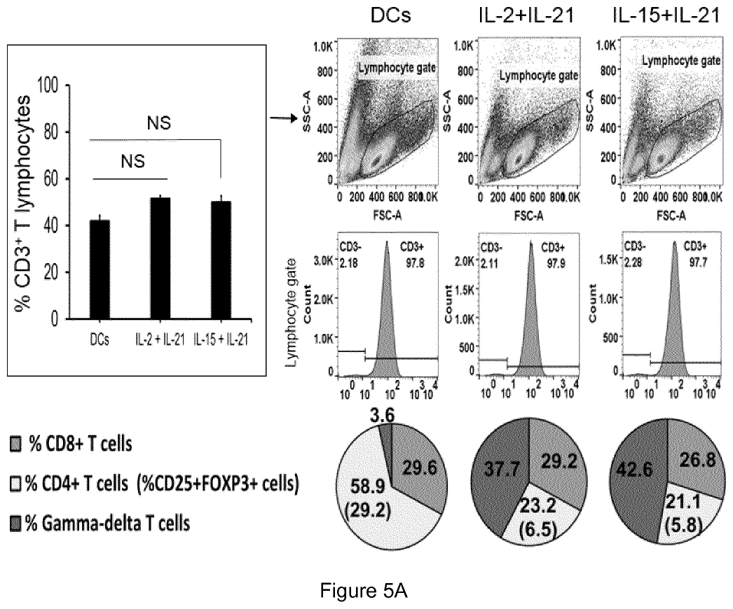

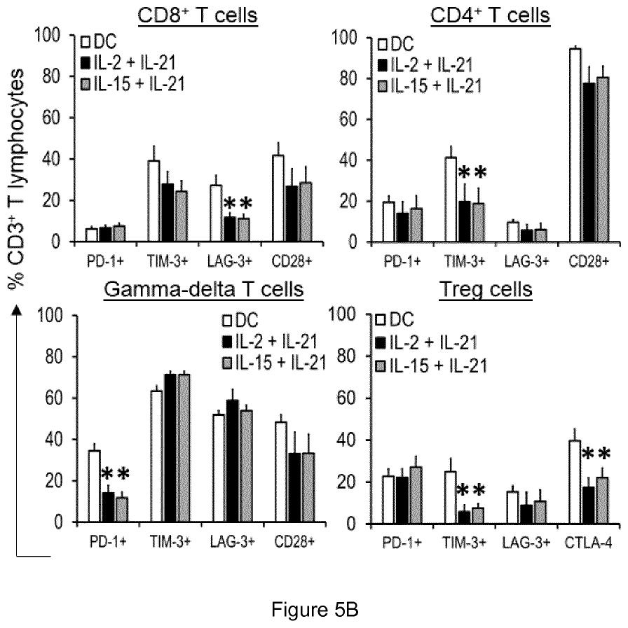

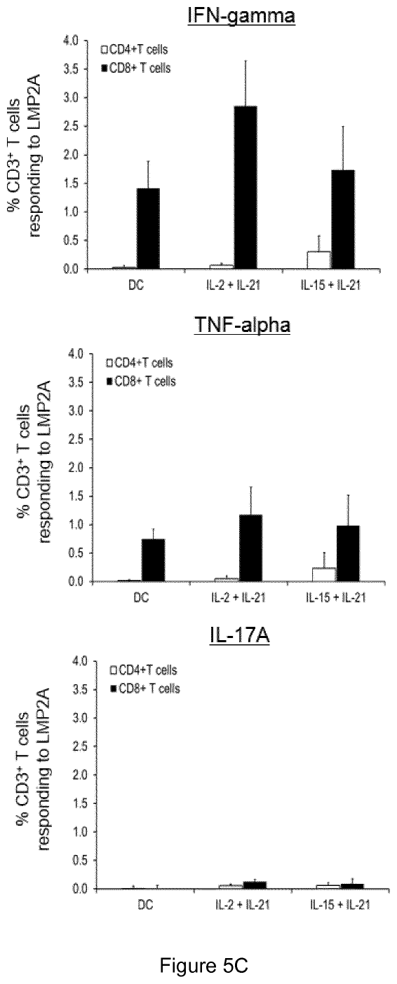

To further evaluate the phenotype and cytokine profile of the T cells stimulated by antigen-pulsed γ9δ2 T cells, we cocultured PBLs with γ9δ2 T cells pulsed with pooled overlapping peptides of EBV-LMP2A. By comparing to monocyte-derived DCs pulsed with the same peptide pool, we observed that peptide-pulsed γδ T cells stimulated an overall higher, though not significant, number of CD3⁺ T lymphocytes (FIG. 5A). We further characterized the % of CD4⁺, CD8⁺ and Treg cells in the CD3⁺ T lymphocyte population and found that peptide-pulsed γδ T cells stimulated almost equal % of CD4⁺ and CD8⁺ T cells (FIG. 5A, pie chart). On the other, the peptide-pulsed monocyte-derived DCs stimulated a predominantly CD4⁺ T cell population which approximately half (i.e. 29.2% of the CD3⁺ CD4⁺ T cells) were CD4⁺ CD25⁺ FOXP3⁺ Tregs (FIG. 5A, pie chart). In contrast, much lower % of CD4⁺ CD25⁺ FOXP3⁺ Tregs (6.5% and 5.8%) was detected when cocultured with IL-2+IL-21 and IL-15+IL-21 generated γδ T cells. Interestingly, the peptide-pulsed γδ T cells persisted in the cocultures and represented more approximately half of the CD3⁺ T lymphocyte population (FIG. 5A, pie chart). We further characterized the exhaustion phenotype of the stimulated CD3⁺ T lymphocytes and found that DCs activated significantly more LAG-3⁺ CD8⁺ T cells and TIM-3⁺ CD4⁺ T cells compared to γδ T cells (FIG. 58). Tregs activated by DCs also expressed higher level of TIM-3 as well as CTLA-4. We further observed that a high % of peptide-pulsed γδ T cells underwent exhaustion as showed their PD-1, TIM-3 and LAG-3 expressions (FIG. 5B, grey and black bars). Similar observations were made in the cocultures where γδ T cells in PBLs that were activated by peptide-pulsed DCs (FIG. 51, white bars). Furthermore, we showed that more IFN-γ secreting CD8⁺ T cells were specific to the EBV-LMP2A pooled peptides following stimulation γδ T cells compared to with DCs (FIG. 5C). Overall, these results suggested that peptide-pulsed γδ T cells (whether generated with IL-2+IL-21 or IL-15+IL-21) were more efficient than monocyte-derived DCs in stimulating more antigen-specific IFN-γ secreting CD8⁺ and CD4⁺ T cells, as well as CD8⁺ and CD4⁺ T cells that were less exhausted in phenotype and less Tregs. These results also suggested that γδ T cell-based therapy could highly benefit from immune checkpoint blockage of TIM-3, LAG-3 and/or CTLA-4 to augment the anti-tumor activities of γδ T cells, CD8⁺ and CD4⁺ T cells.

Discussion

Compelling evidence from clinical and preclinical studies now shows that γδ T cells play an important role in tumor surveillance through active surveying and elimination of transformed cells in the body. γδT cells exhibit unique antigen specificities compared to αβ CD4⁺ and CD8⁺ T cells that recognize tumor-derived peptides presented by professional antigen-presenting cells such as DCs; γδ T cells show diverse antigen specificity towards phosphoantigens (e.g. IPP), self-derived stress-induced ligands on tumor cells (e.g. MICA, MICB, ULBP and HSP) and lipids. They also recognize protein antigens via their γδ TCR (reviewed in 1). Recently, they have been shown to display antigen-presentation and the ability to activate CD4⁺ and CD8⁺ T cells (39, 40). Promising results have been observed in B cell leukemia, prostate and renal cell carcinoma patients whereby some of them achieved partial remission and stable diseases after Vγ9Vδ2 T cell treatment (18-20). Thus, these findings strongly support the rationale of Vγ9Vδ2 T cell-based tumor immunotherapy.

As the procedures for ex vivo generation of Vγ9Vδ2 T cell are highly variable and difficult to compare in many clinical trials even within a given clinical setting or disease, there is a strong need to define a set of robust criteria for producing Vγ9Vδ2 T cells that are highly immunogenic and effective for the clinic. We were the first to describe and define a set of important culture parameters for large-scale clinical production of Vγ9Vδ2 T cells that could highly influence the quality of their phenotype, cytokine profile and anti-tumor functions.

First, we evaluated two clinical grade media (i.e. Click's and Optimizer T cell media) in combination with different serum (i.e. pooled human AB serum and defined FBS) for culturing Vγ9Vδ2 T cells.

We chose these two cell culture media because they have been used extensively for culturing CD4⁺ and CD8⁺ T cells in the clinics, in particular, Optimizer T cell medium could be used serum-free. However, no study have evaluated the effect of these culture media on Vγ9Vδ2 T cells. We chose to supplement the cell culture with pooled human AB serum (2% and 5%) or defined FBS (10%) because both types of sera are already in clinical use and are compatible for producing CD4⁺ and CD8⁺ T cells. In our Phase I/II clinical trial in NPC, we had successfully used Click's media supplemented with 10% defined FBS for large-scale production of EBV-specific CD8⁺ CTLs. Thus, this positive experience led us to select Click's medium and defined FBS as two cell culture components to be evaluated in this study. When used serum-free, we found that Optimizer T cell medium and not Click's medium was able to support Vγ9Vδ2 T cell growth. The yield and % purity of Vγ9Vδ2 T cells generated from PBMCs were significantly increased when pooled human AB serum or defined FBS was added. We also determined that 10% defined FBS was superior to 2% and 5% pooled human AB serum in supporting the rapid proliferation of Vγ9Vδ2 T cells. Hence, we chose Optimizer T cell media supplemented with 10% defined FBS as the optimal medium for further evaluation.

IL-2, IL-15, IL-7, IL-18 and IL-21 are well-studied cytokines for CD4⁺ and CD8⁺ T cell growth and functions. Amongst these cytokines, only IL-2 is used widely in the clinics for γδ T cell proliferation. We evaluated the above cytokines either individually or in combinations on γδ T cell growth. It was noted that IL-18 and IL-21 were not known to support CD4⁺ and CD8⁺ T cell growth, therefore we did not assessed them individually in this study. Similar to reported studies, IL-2 alone was able to induce strong proliferation of Vγ9Vδ2 T cells. Vγ9Vδ2 T cell yield and % purity were increased when IL-2 was used in combination with IL-7 or IL-21. On the other hand, the addition of IL-15 or IL-18 to IL-2 adversely reduced the growth of Vγ9Vδ2 T cells. The use of IL-15 alone or in combinations with IL-7 and IL-21 also supported a stronger Vγ9Vδ2 T cell proliferation. Similarly, the addition of IL-2 or IL-18 led to reduced Vγ9Vδ2 T cell yield and purity. Notably, IL-21 synergized with IL-2 and IL-15 to significantly enhance the yield and % purity of Vγ9Vδ2 T cells. The contrasting effects of IL-18 and IL-21 on Vγ9Vδ2 T cell proliferation were outside the scope of this study. As IL-21 is known to support CD4⁺ T cell differentiation, we speculated that Vγ9Vδ2 T cells might share similar properties as CD4⁺ T cell and hence IL-21 exerted a beneficial effect on their growth.

We found that all the Vγ9Vδ2 T cells generated exhibited both antigen presentation and effector phenotypes. This is important as it suggested that these Vγ9Vδ2 T cells have the ability to perform both antigen-presentation and direct tumor cytolysis functions. We found that the Vγ9Vδ2 T cells generated, regardless of cytokine combinations, were highly capable of producing IFN-γ and TNF-α. This finding is important as IFN-γ and TNF-α exert important anti-tumor functions and are required for activating DCs, CD4⁺ and CD8⁺ T cells. Thus, this suggested that the generated Vγ9Vδ2 T cells are highly capable of activating these immune cells after administration.

Interestingly, we observed that the use of IL-15 (alone or in combination with IL-7 or IL-21) in the cell culture assisted in generating a higher percentage of Vγ9Vδ2 T cells that produced IFN-γ and TNF-α simultaneously upon PMA and ionomycin activation. Thus, the use of IL-15 not only improved the yield and purity of Vγ9Vδ2 T cells, it also helped to enhance their proinflammatory cytokine secretions. Encouragingly, very low % of Vγ9Vδ2 T cells produce IL-17 and IL-10, indicating that they would not actively support tumor and T regulatory cell growth.

Similar to CD8⁺ CTLs, the ex vivo generated Vγ9Vδ2 T cells used granzymes A and B, perforin and granulysin to target and lyse tumor cells. We also noted that the Vγ9Vδ2 T cells reacted most strongly against C666-1 NPC line which actively expressed EBV-related antigen, indicating that Vγ9Vδ2 T cell-based immunotherapy might be particularly useful against viral-related cancers. In addition, we also found that the Vγ9Vδ2 T cells generated were superior to monocyte-derived ‘classical Day 7’ DCs in simulating the proliferation of naïve CD4⁺ and CD8⁺ T cells. In our large-scale study, we further showed that peptide-pulsed γδ T cells (whether generated with IL-2+IL-21 or IL-15+IL-21) were more efficient than monocyte-derived DCs in stimulating more antigen-specific IFN-γ secreting CD8⁺ and CD4⁺ T cells, as well as CD8⁺ and CD4⁺ T cells that were less exhausted in phenotype and fewer Tregs.

In conclusion, we had optimized different culture parameters for large-scale Vγ9Vδ2 T cell production. We evaluated important cell culture parameters that could highly influence the quality of Vγ9Vδ2 T cell phenotype, cytokine prolife, direct tumor cytolysis and antigen presentation for activating anti-tumor CD4⁺ and CD8⁺ T cells. We determined that Optimizer T cell medium supplemented with 10% defined FBS, IL-15 (10 ng/ml) and IL-21 (30 ng/ml) was optimal for generating more than 25 million Vγ9Vδ2 T cell with a purity of 285% from a starting population of 10 million cryopreserved PBMCs. We also determined that the Vγ9Vδ2 T cell generated under this culture condition exhibited desirable antigen-presentation and effector phenotypes, were highly tumor cytolytic and stimulated strong naïve CD4⁺ and CD8⁺ T cell proliferation. The ex vivo generated γδ T cells stimulated more IFN-γ antigen-specific CD8⁺ T cells as well as less exhausted T cells and fewer Tregs compared to DCs in our experimental system. γδ T cell-based therapy could highly benefit from immune checkpoint blockage of TIM-3, LAG-3 and/or CTLA-4 to augment the anti-tumor activities of γδ T cells, CD8+ and CD4+ T cells. This is the first study that provides important insight into the anti-tumor properties of Vγ9Vδ2, as well as essential preclinical data for the development of potent Vγ9Vδ2 T cell-based immunotherapy which is applicable to many tumor types.

### REFERENCES

1. Silva-Santos B, Serre K, Norell H. γδ T cells in cancer. (2015) Nat Rev Immunol 15:683-91.
2. Parker C M, Groh V, Band H, et al. (1990). Evidence for extrathymic changes in the T cell receptor γ/δ repertoire. J Exp Med 1990; 171:1597-612.
3. Morita C T, Jin C, Sarikonda G, et al. (2007). Nonpeptide antigens, presentation mechanisms, and immunological memory of human Vγ2Vδ2 T cells: discriminating friend from foe through the recognition of prenyl pyrophosphate antigens. Immunol Rev 2007; 215:59-76.
4. Tanaka, Y., Morita, C. T., Nieves, E., et al. (2015) Natural and synthetic non-peptide antigens recognized by human γδ T cells. Nature 375:155-158.
5. Morita, C. T. et al. (1995) Direct presentation of nonpeptide prenyl pyrophosphate antigens to human γδ T cells. Immunity 3:495-507.
6. Thompson K, Rogers M J. (2004). Statins prevent bisphosphonate-induced γδ-T-cell proliferation and activation in vitro. J Bone Miner Res 19:278-88.
7. Gober H J, Kistowska M, Angman L., et al. (2003). Human T cell receptor γδ cells recognize endogenous mevalonate metabolites in tumor cells. J Exp Med 197:163-8.
8. Zheng B J, Chan K W, Im S., et al. (2001). Anti-tumor effects of human peripheral γδ T cells in a mouse tumor model. Int J Cancer 92:421-5.
9. Guo B L, Liu Z, Aldrich W A, Lopez R D. (2005). Innate anti-breast cancer immunity of apoptosis-resistant human γδ-T cells. Breast Cancer Res Treat 93:169-75.
10. Kenna T, Golden-Mason L, Norris S., et al. (2004). Distinct subpopulations of γδ T cells are present in normal and tumor-bearing human liver. Clin Immunol 113:56-63.
11. Ferrarini M, Heltai S, Pupa S M., et al. (1996). Killing of laminin receptor-positive human lung cancers by tumor infiltrating lymphocytes bearing γδ+ T-cell receptors. J Natl Cancer Inst 88:436-41.
12. Kobayashi H, Tanaka Y, Yagi J., et al. (2001). γ/δ T cells provide innate immunity against renal cell carcinoma. Cancer Immunol Immunother 50:115-24.
13. Viey E, Fromont G, Escudier B., et al. (2005). Phosphostim-activated γδ T cells kill autologous metastatic renal cell carcinoma. J Immunol 174:1338-47.
14. Kabelitz D, Wesch D, Pitters E, et al. (2004). Characterization of tumor reactivity of human Vγ9Vδ2 γδ T cells in vitro and in SCID mice in vivo. J Immunol 173:6767-76.
15. Liu Z, Guo B L, Gehrs B C, et al. (2005). Ex vivo expanded human V γ9Vδ2+γδ-T cells mediate innate antitumor activity against human prostate cancer cells in vitro. J Urol 173:1552-56.
16. Schilbach K E, Geiselhart A, Wessels J T, et al. (2000). Human γδ T lymphocytes exert natural and IL-2-induced cytotoxicity to neuroblastoma cells. J Immunother 23:536-48.
17. Malkovska V, Cigel F K, Armstrong N, et al. (1992). Antilymphoma activity of human γδ T cells in mice with severe combined immune deficiency. Cancer Res 52:5610-16.
18. Wilhelm M, Kunzmann V, Eckstein S, et al. (2003). γδ T cells for immune therapy of patients with lymphoid malignancies. Blood 102:200-06.
19. Dieli F, Vermijlen D, Fulfaro F., et al. (2007). Targeting human γδ T cells with zoledronate and interleukin-2 for immunotherapy of hormone-refractory prostate cancer. Cancer Res 67:7450-7.
20. Deniger D C, Moyes J S, Cooper U. (2014). Clinical applications of gamma delta T cells with multivalent immunity. Front Immunol 5:636.
21. Chia W K, Teo M, Wang W W, et al. (2014). Adoptive T-cell transfer and chemotherapy in the first-line treatment of metastatic and/or locally recurrent nasopharyngeal carcinoma.

Mol Ther 22: 132-9.
22. Toe J G, Pellegrini M, Mak T W. (2013). Promoting immunity during chronic infection—the therapeutic potential of common gamma-chain cytokines. Mol Immunol 56:38-47.
23. Cox M A, Kahan S M, Zajac A J. (2013). Anti-viral CD8 T cells and the cytokines that they love. Virology 435:157-169.
24. Weng N P, Liu K, Catalfamo M., et al. (2002). IL-15 is a growth factor and an activator of CD8 memory T cells. Ann N Y Acad Sci. 975:46-56.
25. Li W, Kubo S, Okuda A., et al. (2010). Effect of IL-18 on expansion of gammadelta T cells stimulated by zoledronate and IL-2. J Immunother 33:287-96.
26. Thedrez A, Harty C, Morice A., et al. (2009). IL-21-mediated potentiation of antitumor cytolytic and proinflammatory responses of human V gamma 9V delta 2 T cells for adoptive immunotherapy. J Immunol. 182:3423-31.
27. Wu H H, Hwang-Verslues W W, Lee W H, et al. (2015). Targeting IL-17B-IL-17RB signaling with an anti-IL-17RB antibody blocks pancreatic cancer metastasis by silencing multiple chemokines. J Exp Med. 212:333-349.
28. Wang D Z, Wand H B, Brown J, et al. (2006). CXCL1 induced by prostaglandin E2 promotes angiogenesis in colorectal cancer. J Exp Med. 203:941-951.
29. Acharyya S, Oskarsson T, Vanharanta S, et al. (2012). A CXCL1 Paracrine Network Links Cancer Chemoresistance and Metastasis. Cell. 150:165-178.
30. Walch-Rückheim B, Mavrova R, Henning M, et al. (2015). Stromal Fibroblasts Induce CCL20 through IL6/C/EBPβ to Support the Recruitment of Th17 Cells during Cervical Cancer Progression. Cancer Res. 75:5248-5259.
31. Loetscher P, Seitz M, Clark-Lewis I, et al. (1996). Activation of NK cells by CC chemokines. Chemotaxis, Ca2+ mobilization, and enzyme release. J Immunol. 156:322-327.
32. Harlin H, Meng Y, Peterson A C, et al. (2009). Chemokine Expression in Melanoma Metastases Associated with CD8+ T-Cell Recruitment. Cancer Res. 69:3077-3085.
33. Molon B, Ugel S, Del Pozzo F, et al. (2011). Chemokine nitration prevents intratumoral infiltration of antigen-specific T cells. J Exp Med. 208:1949-1962.
34. Fridlender Z G, Buchlis G, Kapoor V, et al. (2010). CCL2 Blockade Augments Cancer Immunotherapy. Cancer Res. 70:109-118.
35. Castellino F and Germain R N. (2007). Chemokine-Guided CD4+ T Cell Help Enhances Generation of IL-6RahighIL-7Rahigh Prememory CD8+ T Cells. J Immunol. 178:778-787.
36. Hickman H D, Li L, Reynoso G V, et al. (2011). Chemokines control naïve CD8+ T cell selection of optimal lymph node antigen presenting cells. J Exp Med. 208:2511-2524.
37. Mikucki M E, Fisher D T, Matsuzaki J, et al. (2015). Non-redundant requirement for CXCR3 signalling during tumoricidal T-cell trafficking across tumour vascular checkpoints. Nat Commun. 25:7458.
38. Sharma S, Yang S C, Hillinger S, et al. (2003). SLC/CCL21-mediated anti-tumor responses require IFNγ, MIG/CXCL9 and IP-10/CXCL10. Mol Cancer. 15:22.
39. Brandes M, Willimann K, Moser B. Professional antigen-presentation function by human gammadelta T Cells. (2005). Science 309:264-268.
40. Meuter S, Eber M, Moser B. (2010). Prolonged antigen survival and cytosolic export in cross-presenting human γδ T cells. Proc Natl Acad Sci USA. 107:8730-8735.

### Example 2: In Vivo Experiments in Mice

Anticancer activity of adoptively transferred γδ T cells was analysed in vivo in experiments performed in a mice.

Experiment 1

Tumours were established by subcutaneous injection of mice with 5×10⁶ lymphoblastoid cell line cells (LCLs) on Day 0.

Mice were divided into groups of 3-4 mice, and assigned to one of four treatment groups a) to d) below:
a) No treatment (mock treatment by injection of basal media)
b) γδ T cells only (designated ‘gd’)−1×10⁶ cells per mouse, per treatment
c) CSFE-labelled, pan naïve αβ T cells only (designated ‘ab’)−1×10⁶ cells per mouse, per treatment
d) Initial administration of γδ T cells and CSFE-labelled, pan naïve αβ T cells in a 1:1 ratio (Treatment 1), followed by administration of γδ T cells (Treatments 2 and 3)−1×10⁶ cells per mouse, per treatment.

The γδ T cells used in Experiment 1 were prepared as described in Example 1.

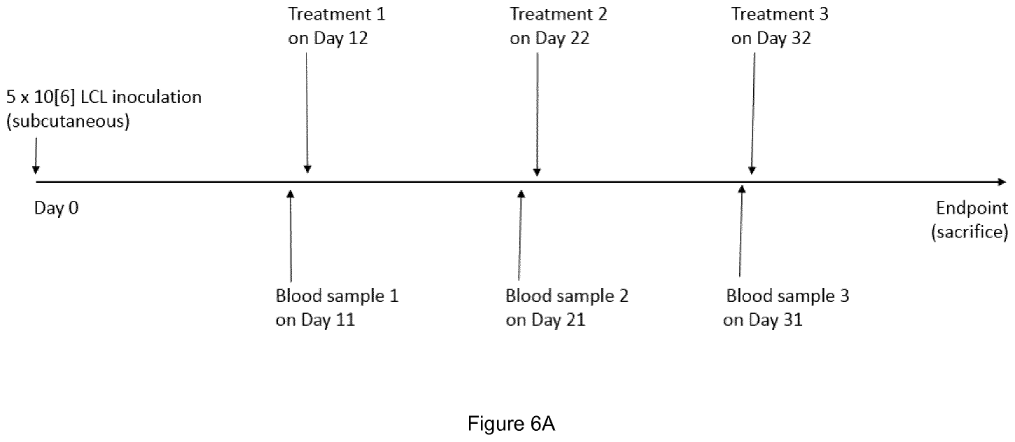

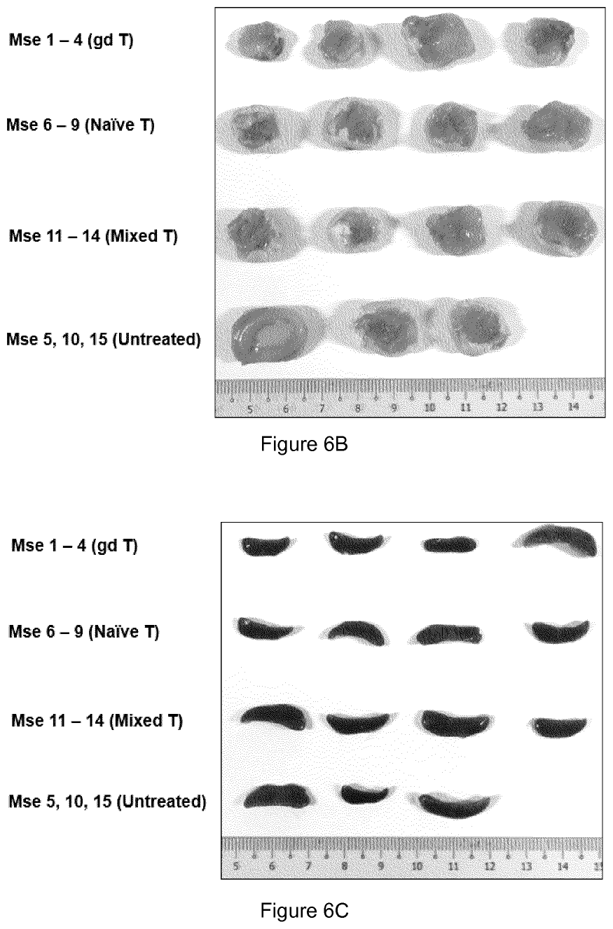

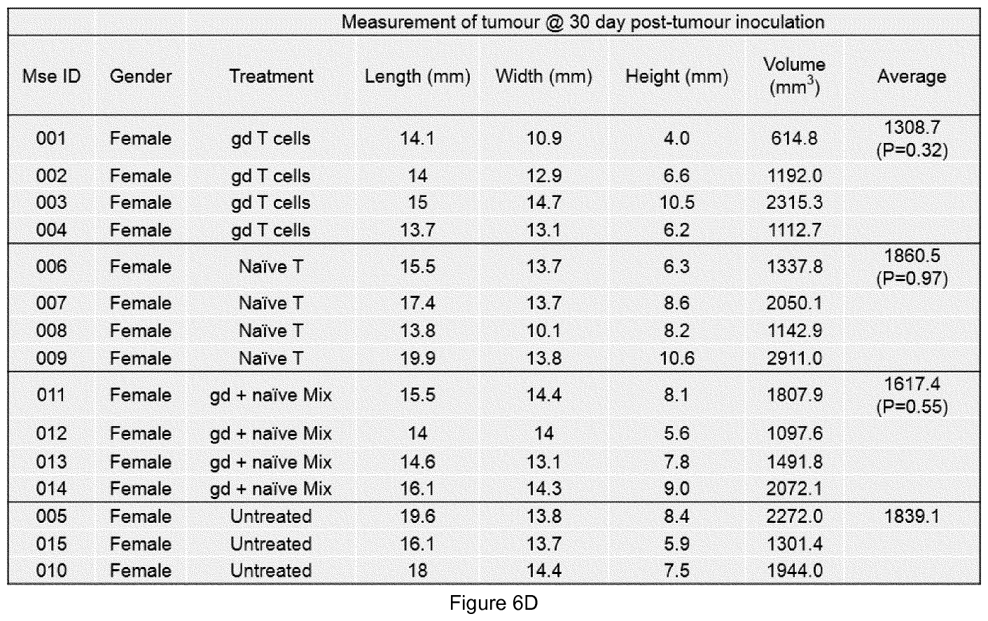

All treatments were administered intratumorally from Day 12, every 10 days, and blood samples were obtained prior to every treatment. A schematic representation of the procedures for Experiment 1 is shown in FIG. 6A.

At the end of the experiment the tumors and spleens were harvested and analysed (FIGS. 6B and 6C).

The size and volume measurements of the tumors harvested from the mice are shown in the Table of FIG. 6D. Tumors obtained from mice which received treatment with γδ T cells were smaller and had a reduced volume as compared to the tumors obtained from mice which were untreated, or mice which were treated with naïve ap T cells. The greatest reduction in tumor size and volume (as compared to the untreated control) was observed in mice from treatment group b), which were treated with γδ T cells only.

Administration of γδ T cells was therefore demonstrated to have an antitumor effect.

Experiment 2

Tumours were established by subcutaneous injection of mice with 2×10⁶ LCLs on Day 0.

Mice were divided into groups of 3-4 mice, and assigned to one of five treatment groups a) to e) below:
a) No treatment (mock treatment by injection of basal media), administered intratumorally
b) γδ T cells only—2×10⁶ cells per mouse, per treatment, administered subcutaneously
c) γδ T cells only—2×10⁶ cells per mouse, per treatment, administered intravenously
d) CSFE-labelled, pan naïve αβ T cells only—2×10⁶ cells per mouse, per treatment administered intratumorally
e) Initial administration of γδ T cells and CSFE-labelled, pan naïve αβ T cells in a 1:1 ratio (Treatment 1), followed by administration of γδ T cells (Treatments 2 and 3)—2×10 cells per mouse, per treatment, administered intratumorally.

The γδ T cells used in Experiment 2 were prepared as described in Example 1.

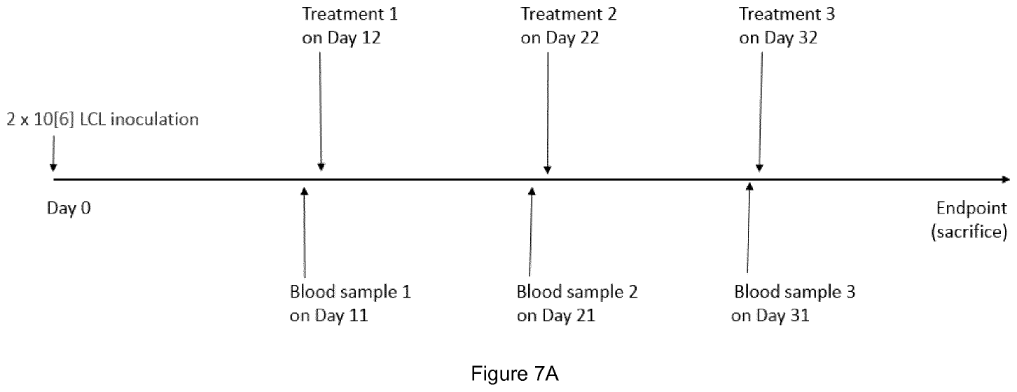

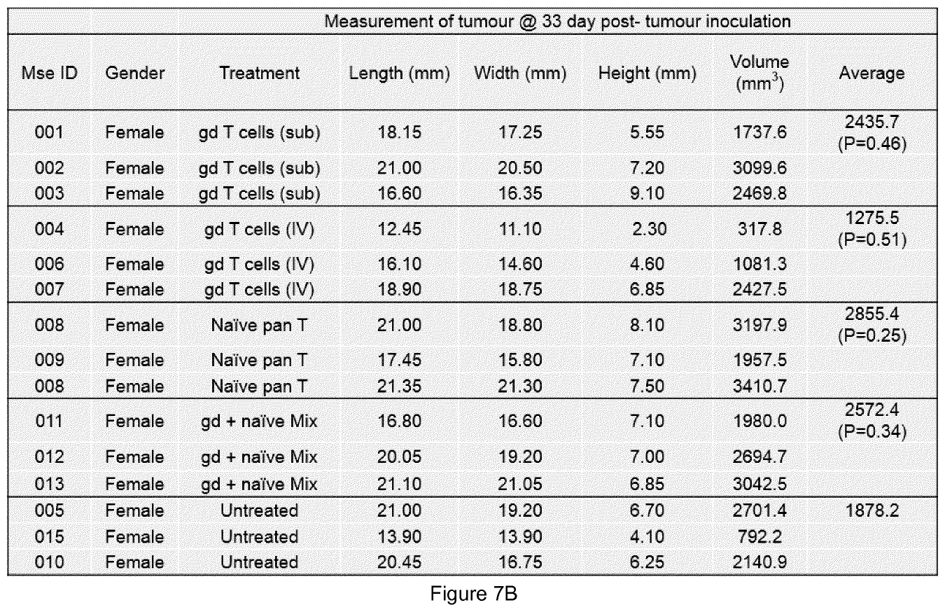

Treatments were administered from Day 12, every 10 days, and blood samples were obtained prior to every treatment. A schematic representation of the procedures for Experiment 2 is shown in FIG. 7A.

At the end of the experiment the tumors and spleens were harvested and analysed. The size and volume measurements of the tumors harvested from the mice are shown in the Table of FIG. 7B.

Tumors obtained from mice which received treatment with γδ T cells via intravenous administration (treatment group c) were smaller and had a reduced volume as compared to the tumors obtained from mice of the other treatment groups.

Intravenous administration of γδ T cells was therefore demonstrated to have an antitumor effect.

Experiment 3

In a further experiment, tumours are established by subcutaneous injection of mice with 5×10⁵ LCLs on Day 0.

Mice are divided into groups of 3 mice, and assigned to one of five treatment groups 1) to 5) below:
1) No treatment (mock treatment by injection of basal media)
2) 10×10⁶ γδ T cells only+100 μg/kg zoledronic acid per mouse, per treatment
3) 10×10⁶ peripheral blood lymphocytes (PBLs)+100 μg/kg zoledronic acid per mouse, per treatment
4) 10×10⁶ cells from a coculture of γδ T cells and PBLs+100 μg/kg zoledronic acid per mouse, per treatment
5) 100 μg/kg zolendronic acid per mouse, per treatment

All treatments are administered intravenously from Day 12, every 10 days, and blood samples are obtained prior to every treatment.

Zoledronic acid is obtained from Sigma Aldrich and is diluted in basal media prior to administration.

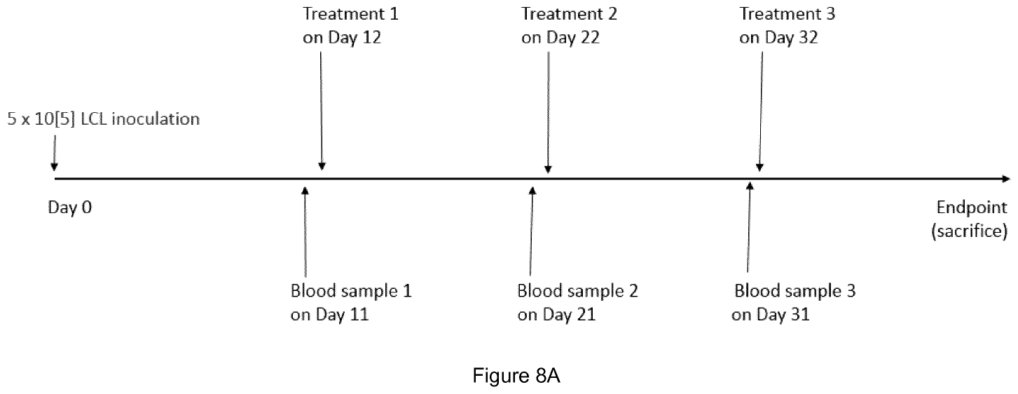

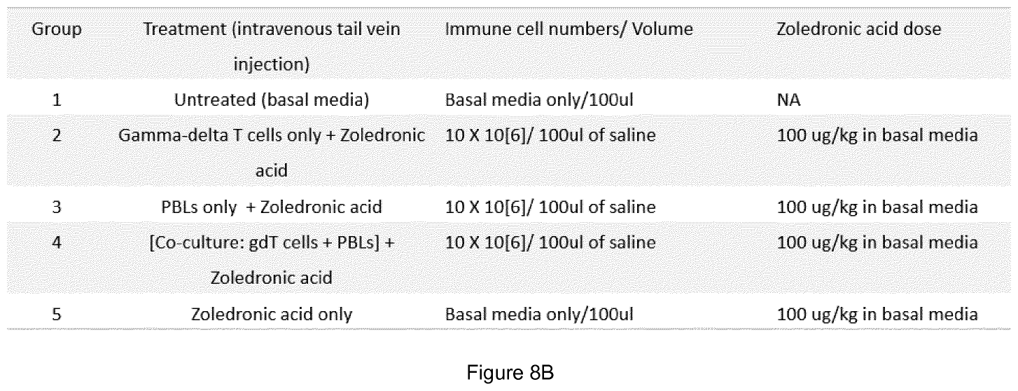

A schematic representation of the procedures for Experiment 3 is shown in FIG. 8A, and a summary of the treatments for each treatment group is shown in FIG. 88.

The γδ T cells used in Experiment 3 are prepared as described in Example 1, with the following variations:
(i) 10 μM is added to the cultures on days 1, 3 and 5
(ii) 400 units/ml IL-2 and 60 ng/ml of IL-21 are added to the cultures on days 1, 2, 5 and 8
(iii) The γδ T cells are isolated on day 11.

It is expected that combination treatment with γδ T cells and zoledronic acid (e.g. treatment group 2) will display greater antitumor activity as compared to treatment with zoledronic acid alone (treatment group 5).

1. A method for generating or expanding gamma delta T cells, the method comprising culturing peripheral blood mononuclear cells (PBMCs) in the presence of IL2 and IL21.

2. A method for generating or expanding gamma delta T cells, the method comprising culturing PBMCs in the presence of 112 and IL18.

3. A method for generating or expanding gamma delta T cells, the method comprising culturing PBMCs in the presence of IL15.

4. The method according to claim 3, wherein the PBMCs are cultured in the presence of IL15 and IL21.

5. The method according to claim 4, wherein the PBMCs are cultured in the presence of IL15, IL 21 and IL18.

6. A method for generating or expanding gamma delta T cells, the method comprising culturing PBMCs in the presence of IL21.

7. The method according to claim 6 wherein the PBMCs are cultured in the presence of IL21 and IL2 and/or IL15.

8. The method according to any one of the preceding claims wherein the gamma delta T cells are Vδ2 T cells.

9. The method according to claim 8, wherein the gamma delta T cells are Vγ9Vδ2 T cells.

10. The method according to any one of the preceding claims, wherein the method comprises culturing the PBMCs in culture medium supplemented with serum.

11. The method according to claim 10 wherein the culture medium is supplemented with 10% serum.

12. The method according to any one of the preceding claims the PBMCs are cultured in OpTimizer® T cell media.

13. The method according to claim 10 or claim 11 wherein the serum is human AB serum or defined FBS.

14. The method according to any one of claims any one of the preceding claims, wherein the method generates a population of gamma delta T cells that is at least 60% gamma delta T cells, preferably at least 70% gamma delta T cells.

15. The method according to any one of the preceding claims, wherein the gamma delta T cells exhibit antigen presentation and effector phenotypes.

16. A gamma delta T cell generated using a method according to any one of the preceding claims.

17. The gamma delta T cell according to claim 16 for use in medicine.

18. The gamma delta T cell according to claim 16 for use in a method of adoptive T cell therapy.

19. A gamma delta T cell that expresses a higher level of at least one marker selected from HLA-ABC, HLA-DR, CD80, CD83, CD86, CD40 and ICAM-1 than a gamma delta T cell that has been generated in the presence of IL2 alone.

20. A gamma delta T cell that expresses a higher level of at least one marker selected from CCR5, CCR6, CCR7, CD27 and NKG2D than a gamma delta T cell that has been generated in the presence of IL2 alone.

21. A cell culture comprising gamma delta T cells, media, and
IL2 and IL21;
IL15;
IL15 and IL21;
IL2 and IL18;
IL15, IL18 and IL21.
IL2 and IL7;
IL2 and IL15;
IL2, IL18 and IL21;
IL15 and IL7; or
IL15 and IL18.

22. The cell culture according to claim 21, further comprising serum, preferably 10% serum.

23. A method for generating or expanding a population of antigen-specific T cells, comprising stimulating T cells by culture in the presence of gamma delta T cells generated/expanded according to the method of any one of claims 1 to 15 presenting a peptide of the antigen.

24. An antigen-specific T cell according generated using the method according to claim 23.

25. The antigen-specific T cell according to claim 24 for use in medicine.

26. The antigen-specific T cell according to claim 25 for use in a method of adoptive T cell therapy.

27. A method of treating or preventing a disease or disorder in a subject, comprising:
(a) isolating PBMCs from a subject;
(b) generating or expanding a population of gamma delta T cells according to the method of any one of claims 1 to 15, and;
(c) administering the gamma delta T cells to a subject.

28. A method of treating or preventing a disease or disorder in a subject, comprising:
(a) isolating PBMCs from a subject;
(b) generating or expanding a population of gamma delta T cells according to the method of any one of claims 1 to 15;
(c) generating or expanding a population of antigen-specific T cells by a method comprising stimulating T cells by culture in the presence of gamma delta T cells generated/expanded according to (b) presenting a peptide of the antigen; and
(d) administering the antigen-specific T cells to a subject.
